(19) United States
(12) Patent Application Publication
(10) Pub. No.: US 2005/0271653 A1
(43) Pub. Date: Dec. 8, 2005

(54) Methods and devices for targeting a site in a mammal and for removing species from a mammal

(76) Inventor: Meir Strahilevitz, Seattle, WA (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 10/511,818

(22) PCT Filed: Apr. 23, 2003
(86) PCT No.: PCT/US03/12602

§ 371 Date: Jul. 7, 2005

Related U.S. Application Data
(60) Provisional application No. 60/374,715, filed on Apr. 23, 2002.
(60) Provisional application No. 60/381,118, filed on May 17, 2002.
(60) Provisional application No. 60/397,111, filed on Jul. 19, 2002.

(51) Int. Cl. A61K 39/395

(52) U.S. Cl. 424/140.1

(57) ABSTRACT

Methods and devices for improved targeting to a site in an organism, particularly to a tumor target site and for extracorporeal affinity adsorption, particularly in the treatment of cancer, atherosclerosis, including coronary artery disease, unstable angina, other acute ischemic syndromes and idiopathic dilated cardiac myopathy. In one aspect of the invention, a combination is provided comprising an extracorporeal device (1) having contained therein a binding compound (11) bound to a carrier (9), the binding compound having affinity for a binding partner, and a plurality of affinity binders (15), each of said affinity binders comprising a first portion (19) comprising the binding partner and a second portion (17) adapted to bind selectively with a species, the second portions of each of said affinity binders differing from each other.

### CROSS-REFERENCE TO RELATED APPLICATIONS

This application claims priority under 35 U.S.C. §119(e) of U.S. Provisional application 60/374,715, filed 23 Apr. 2002, 60/381,118, filed 17 May 2002, and 60/397,111, filed 19 Jul. 2002.

### TECHNICAL FIELD

The field of the invention is targeting of ligands to a site in an organism, particularly a cancer site, by utilizing adsorbents with selective or specific affinity to chemical species, wherein the adsorption of at least one molecule is associated with improved targeting to the site in the body, such as a tumor site and increased concentration of the ligand in the targeted site, relative to its concentration in the rest of the body.

The present invention relates to methods and devices for improved targeting to a site in an organism, particularly to a tumor target site and for extracorporeal affinity adsorption, particularly in the treatment of cancer, atherosclerosis, including coronary artery disease, unstable angina, other acute ischemic syndromes and idiopathic dilated cardiac myopathy.

The targeting methods and devices include methods and devices for improved targeting of ligands wherein the ligand comprises a targeting molecule, to a site in an organism, particularly a cancer site, comprising affinity binding of at least one of the group comprising of tumor circulating antigen, antibody specific to tumor antigen, complex of tumor circulating antigen and antibody to tumor antigen and antibodies specific to at least one constituent of the ligand.

The extracorporeal affinity adsorption methods and devices include extracorporeal affinity adsorption and other affinity binding treatment methods and devices for atherosclerosis and in particular coronary artery disease, particularly for unstable angina and other acute ischemic syndromes, for idiopathic dilated cardiac myopathy and for intoxication with toxins, in particular bacterial exotoxins.

The affinity adsorption methods and devices are themselves novel and find use in other fields.

The following list of some of the abbreviations used in the application does not include all the abbreviations used; additional abbreviations are defined in the text, but not included in the list:
TC: tumor compartment.
BC: blood compartment.
HC: healthy organs compartment.
IF: interstitial fluid.
TL: treatment ligand. As used in the current application a “treatment ligand” (TL) is a drug, radioactive molecule, radioactive atom, radioactive ion, or a cell, when used as a therapeutic agent, when the TL, as herein defined, is adsorbed to an adsorbent (such as an adsorbent in an extra corporeal device), the TL is still referred to as “ligand”, in accordance with the meaning of the use “ligand” in the current application.
VL: visualization ligand. The same considerations as above apply to the definition of “visualization ligand” (VL) as used in the current application. Visualization ligand (VL) includes radioactive, magnetic, fluorescent and any other visualization ligands, as known in the art, including, but not limited to the visualization ligands specifically mentioned in the current application. The visualization ligands may include, for example, atoms, ions, and molecules when used as visualization aids in diagnosis or treatment.
CSF: cerebrospinal fluid.
PF: peritoneal fluid.
ICF: intra cellular fluid.
TA: tumor antigen (on tumor cell). It is realized that “tumor antigen” include antigens that are preferentially concentrated on tumor cells, but are also present, in lower concentrations, on normal cells.
CA: circulating tumor antigen (circulating molecules, or molecule complexes, that dissociated from, or other wise, originate from tumor cells, but are not a part of the tumor cell, as they circulate in the blood, or present in other biological fluids in the organism).
NAB: native antibodies: antibodies specific and with affinity to TA and CA.
CEA: carcino embryonic antigen.
AFT: alpha fetoprotein.
CA-NAB: circulating tumor antigen-native antibody complex.
CA2-NAB: CA-NAB, with excess antigen.
CA-NAB2: CA-NAB, with excess antibody.
TAB: targeting antibody (including antibody fragments, including fragments produced by synthesis, including antibody binding site analogs).
ECA: extracorporeal adsorption.
OTP: other targeting proteins or peptides. Whenever “TAB” is mentioned, it includes OTP, unless specifically excluded.
TIF: tumor-targeting inhibitory factors: species that inhibit or interfere with targeting TAB or OTP to the tumor.
TISF: tumor immunity suppressor factors: species that interfere with immune inhibition and/or immune destruction of tumor.
TSF: tumor destruction suppressor factors: species that interfere with tumor inhibition and/or destruction by any mechanism, including, but not limited to immunologic mechanism. TSF includes TISF.
SA: specific antibodies.
EDTA: ethylenediaminetetraacetic acid.
STBR: species to be removed (includes both molecular and cellular species).
BAC: biotin adsorbent column.
AAC: avidin adsorbent column.
LAK: lymphokin activated killer cells.
TIL: tumor infiltrating lymphocytes.
CTC: cytotoxic t cells. MIF: molecular inflammatory factor.
CIF: cellular inflammatory factor.
MCIF: molecular and cellular inflammatory factors (MIF+CIF).
AS: atherosclerosis.
CAD: coronary artery disease.
PASCVD: peripheral atherosclerotic vascular disease.
AIS: acute ischemic syndrome.
MI: myocardial infarction.
UA: unstable angina.
EP: etiology and pathogenesis.
MCS: molecular or cellular species.

“Targeted species” include: targeted visualization ligands and targeted treatment ligands.

“Targeting species” include: targeting molecules, including, but not limited to targeting antibodies and targeting antibody fragments, targeting receptor ligands, including synthetic and analogs thereof, including without limitation all the specific targeting species disclosed in the application.

### BACKGROUND ART

The invention, in one of its aspects, relates to targeting of ligands to a site in an organism, particularly a tumor site, by utilizing adsorbents with selective or specific affinity to chemical species, wherein the adsorption of at least one molecule is associated with improved targeting to the site in the body, such as a tumor site and increased concentration of the ligand in the targeted site, relative to its concentration in the rest of the body.

The adsorbents are primarily incorporated in an extracorporeal device, but the field of the invention is not limited to extra corporeal affinity adsorption or extracorporeal affinity dialysis. (Extracorporeal affinity dialysis is a method involving both dialysis (and/or filtration) and adsorption as detailed in my U.S. Pat. No. 5,753,227, see in particular; column 5, line 40 to column 7, line 44.) At least one of the adsorbents may be administered to the organism, such as by intravenous, intraperitoneal, or other route and by binding of the “adsorbent” to the species, the species is cleared faster then it would otherwise clear from the body of the organism by increased elimination from the body, such as elimination through the kidney or liver or reticulo endothelial system (RES), or by increased metabolism and breakdown or neutralization of the chemical species.

The methods and devices that are describing the field of the invention and are relevant to elements of the invention that is the subject matter of the current application, include the following patents, patent applications and publications, all of which, including the references cited in said documents are incorporated herein by reference: U.S. Pat. No. 4,375,414 and U.S. Pat. No. 4,813,924 and any and all divisional applications or patents of said patents; U.S. Pat. No. 6,039,946, U.S. Pat. No. 5,753,227, U.S. Pat. No. 6,264,623 and published U.S. Patent applications U.S. 2001/0039392 A1, US 2002/0019603 A1 and all their US divisional applications and issued patents and all equivalents and foreign counterparts of said patents and the divisional patent applications and divisional patents of said patents; PCT WO96/37516 and its foreign and US counterparts, and all divisional applications thereof. For abbreviation all these patents and patent applications will be referred to at times as “my patents”. Also incorporated herein by reference are the following patents and publications, including the references cited in them: Nillson et al: U.S. Pat. No. 6,251,394 B1, V. Pimm, Nucl. Med. Biol. Vol. 22, No 2, pp. 1020-1027, 1993, D. A. Goodwin et al, Antibody Immunoconjugates and Radiopharmaceuticals, Vol. 4, number 4, pp. 427-434, 1991, Van Kroonenburgh et al., Nucl. Med. Commun., Vol. 9, pp. 919-930, 1988, M. Gurkavij et al., Cancer Research (Suppl.) Vol. 55, pp. 5874s-5880s, Dec. 1, 1995.

S. Yazdani et al., American Heart J., 1998 August, Vol. 136 (2) pp. 357-61, found data indicating that increased IL-6 is correlated with destabilization of atheroma in coronary arteries. H. Tashiro et al., Coronary Artery Disease, 1997 March, Vol. 8 (3-4), pp. 143-147, found that Macrophage Stimulating Factor MCSF is stimulating and Transforming Growth Factor Beta is (TGF-Beta) is inhibiting the process of AS. D. A. Smith et al., Circulation, 2001, Aug. 14, Vol. 104 (7) pp. 746-9, reported protective effect of IL-10 in AS.

G. Luizo et al, Circulation, 1999, Nov. 23, Vol. 100(21) pp. 2135-9 reported in UA increase in CD4+ CD28null cells associated with INF gamma production that was associated with persistent antigenic stimuli. D. E. Newby and K. A. Fox Br Med Bull 2001, Vol. 59, pp. 69-87, reported that use of anti-inflammatory clopidogrel and glycoprotein IIb/IlIa receptor antagonists had positive effect on UA.

### SUMMARY OF THE INVENTION

The limitations inherent in methods known to date, for the targeting of ligands, such as treatment ligands (TL) and visualization ligands (VL), to a site in an organism, particularly, but not exclusively tumor site, are related in part, to the presence of molecular species in the blood and other body fluids, that compete with or interfere with targeting to the site. The invention provides means to improve targeting by the utilization of affinity-based removal of molecular species that interfere or compete with targeting. As known in the art of targeting ligands to sites in the organism, such sites may be, for example, an area of infarct in the heart, or a specific organ such as the thyroid gland, as examples. The tumor sites, in accordance with the current invention are both solid tumors that are tumors originating from cells outside the blood, the bone marrow and the lymphatic system. Examples of solid tumors are well known and include, for example, colon carcinoma ovarian carcinoma, prostate carcinoma, pancreatic carcinoma as well as sarcomas, as well as brain tumors, such as astrocytoma and glioblastoma as well as “soft tissue tumors” such as leukemias and lymphomas, which originate in the bone, marrow the lymphatic system and the blood.

The treated organism which is a mammal, including, but not limited to a human, is conceptualized in accordance with the present invention to contain three compartments: the tumor compartment (TC), the blood compartment (BC) and the rest of the organism: the healthy tissue and healthy organs compartment (HC), including for example, the liver, kidney, spleen and lymph nodes. It should be realized that some or all of the organs may include tumor cell masses (such as metastases of tumor). As used in the current invention the non-tumor tissue and cells of the organ (kidney and liver for example) will be referred to as HC and the metastatic cells (typically in the form of multiple cells, but may be single cells): are referred to as TC, even though they reside in the kidney or the liver, for example. It is clearly appreciated that the three compartments are in fluid communications, through, but not limited to the interstitial fluid (IF), the capillaries of the blood circulatory system and the fluids of the nervous system, cerebrospinal fluid (CSF) the lymphatic system fluid and peritoneal fluid (PF), for example. Further more, there is also fluid communications between the intracellular fluid (ICF) and the other compartments, including the above fluid compartments, for example the IF. It is also realized, that various chemical and cellular species, such as proteins, peptides, various antigenic and haptenic molecules, whether endogenous to the organism, or administered to the organism (such as TL and VL), various subpopulations of lymphoid cells and macrophages, invading microorganisms such as viruses, bacteria and protozoa, are able to move from one compartment to the other, and that molecular species may be at a steady state balance between the various fluid compartments and TC, BC and HC, meaning that removal of a molecular species from the BC, may for example change the rate of movement of the removed species from the other compartments to the BC. In accordance with the present invention the tumor cells of the present invention contain tumor antigens (TA) and the blood compartment (BC) may contain circulatory tumor antigens (CA). TA is a chemical species comprising an integral part of the tumor cell. CA is a chemical species which is free and does not comprise an integral part of a tumor cell as it circulates in the blood and/or in other biological fluids. It will be realized in accordance with the discussion above, that the CA, being a chemical species, usually will be able to migrate between the various compartments. A CA may be identical in chemical and antigenic structure to TA or it may have chemical and antigenic structure that is similar to but not identical to TA (for example the CA may have different affinity to specific monoclonal antibodies specific to TA then the affinity of same antibodies to TA). The body of the organism may produce antibodies to the TA and/or the CA. These antibodies are produced by the organism, as the result of the immune system in the organism mounting a humoral immune response directed at the TA and/or the CA. These antibodies are referred to as native antibodies (NAB). NAB may act as “Enhancing Antibodies” (K. A. Hellstrom and I. Hellstrom: In: Encyclopedia of Immunology, I. M. Roitt and P. J. Delves Eds. Academic Press, 1998 p. 2440-2445), such antibodies may bind to TA and mask the TA on the tumor cells, or they may participate in inducing suppressor lymphoid cells that inhibit tumor killing by cytotoxic T cells (CTC), thus interfering with induction of cellular immunity to the tumor (afferent inhibition of tumor immunity) or they may block the tumor antigen (TA) and shield the tumor cell from immune destruction by lymphoid cells, such as cytotoxic T cells (CTC), (efferent inhibition of tumor immunity). Examples of TA are given in WO 96/37516 and include carcinoembryonic antigen (CEA), Le(y), and alpha-fetoprotein (AFT). Many other tumor antigens which include tumor specific antigens and developmental antigens (for example ovarian carcinoma CA-125 antigen and CEA antigen, respectively) as well as monoclonal antibodies specific to these antigens are known in the art. (B. J Van den Eynde and A. M. Scott: in Encyclopedia of Immunology, supra pp. 2424-2430.) In the presence of both CA and NAB, at least some of the CA and NAB bind to each other and produced CA-NAB complex. Such complexes may be in “antigen excess” (CA2NAB), wherein the complex will generally have free antigenic sites available to bind additional antibody molecules, or it may be in “antibody excess” (CANAB2), wherein the complex will generally have free antibody binding sites available to bind additional antigen molecules. CA-NAB, was found to play a role in the etiology and pathogenesis of cancer (F. A. Salinas and M. G Hanna, Jr. Eds. Contemporary Topics in Immunobiology, Vol. 15, Immune Complexes and Human Cancer, Plenum Press, 1985) and removal of CA-NAB by plasma exchange can be therapeutic (Immune complexes and Plasma Exchange in Cancer Patients, B. Serrou and C. Rosenfeld, Elsvier/North-Holland Biomedical Press, 1981).

Factors that interfere with targeting TAB-bound VL or TL to a tumor site in an organism include:
(1) Presence of CA or CA2-NAB, that competes with the TA for binding of the TAB.

(2) TAB (bound to VL/TL) or CA-TAB (bound to VL/TL) complex binds to receptors such as Fc Receptors. If TAB is toxic, for example, is bound to a VL or TL that is radioactive or is a toxin or toxic drug, this will lead to increased concentration of toxic VL/TL in healthy organs such as the liver, by binding of the TAB's Fc to Fc receptors on liver cells, when TAB contains Fc, such as when TAB is an intact antibody (it should be realized as mentioned earlier that TAB as defined in the current application may also be an antibody fragment, including synthetic fragment and fragment produced by genetic engineering. Complexes of such fragments with CA, even though they do not have Fc fragment, and do not bind to Fc receptors in the liver, they can still be cleared in the RES in the liver and elsewhere in reticulo endothelial system (RES), by RES cells, such as macrophages.

(3) NAB compete with TAB for binding to TA and may inhibit TH1 Helper Cells' immune response to the cancer.

(4) CA-NAB complex concentrates in the RES, liver and kidney, for example, within the HC and toxic effects on normal organs is induced by this concentration.

(5) Particularly, following repeated administration of heterologus targeting antibody (e.g., mouse monoclonal targeting antibody to human recipient), the recipient may produce antibodies specific to the targeting antibodies. Such antibodies may be specific to the Fc part of the antibody (antiisotypic antibodies) or they may be directed to the Fv fragment (antiidiotypic antibodies). Such anti-targeting antibody antibodies (ATAA) can be produced by the organism; also when the TAB is a chimeric or humanized monoclonal antibody (M. V. Pimm supra, Van Kroonenburgh et al., supra) ATAA may compete with TA for the binding of TAB.

It should be realized that depending on the particular cancer and the individual case, CA, CA-NAB complex, NAB, free ATAA TAB-ATAA complex (after the administration of TAB) may be present in various concentrations in the BC HC and TC. Optimally, particularly when these various concentrations are not known, it may be advantageous to remove or otherwise reduce more than one of the above species, and at least in some situations it may be desirable to remove as many of these species, as possible from the BC and/or HC and/or TC, prior to the administration of the TAB-VL or TAB-TL. Reduction of the amount of one or more of the above species in the BC and/or HC and/or TC, prior to the administration of the TAB-VL or TAB-TL, can preferably be achieved, in accordance with the present invention by extracorporeal adsorption, but may include, in some situations, reduction of one or more of the above species, by the administration to the subject of agents that would increase the breakdown and/or clearance from the body compartments of one or more of the above species (see, for example, D. A. Goodwin, supra).

Alternatively, one or more species that are desired to be removed can be affinity labeled by administration to the subject being treated of the respective affinity binder (such as a specific antibody to the species that are desired to be removed), for example biotinylated antibody or antibodies specific to the species, followed by ECA with a generic device such as an Avidin ECA device when the affinity binder is a biotinylated antibody.

One approach that was used to reduce this interference with targeting is the administration of unlabeled targeting antibody prior to the administration of the labeled targeting antibody. This approach was associated with limited and various success (see for examples: M. Helma et al., Cancer Immunol. Immunother. (1998) 47: 39-46, Beatty, B. G. et al., Cancer Res. 49, 1587-1594, 1989; Ahonen et al., Acta Oncologica (1993), 32, 7/8, pp. 723-7; Schrijvers A. G. H. et al., J Cancer Research, (1993) 53, 4383-04390, Sep. 15, 1993.

Clearly significant improvement in specificity (e.g., to target cell, target tissue or organ, relative to the rest of the body) of targeting is needed. Pre administration of unlabeled targeting antibody, while binding to circulating antigen or to CA2NAB in the blood circulatory system (or in other biological fluid such as for example, cerebrospinal fluid (CSF), lymphatic fluid and peritoneal fluid) and thus reducing the amount of subsequently administered labeled antibody in the blood and/or in the liver, by reducing the amount of circulating antigen that can bind the labeled antibody. However, the pre-administration of unlabeled antibody will also lead to the unlabeled antibody binding to the tumor antigen on tumor cells and by this mechanism will reduce targeting of later administered labeled antibody to the tumor target.

Thus, generally extracorporeal adsorption (ECA) is preferred, in view of the fact that such a method is not associated with administration of additional species to the subject (that may have their own toxicity) and is not associated with increased concentration of TAB, TL or VL in healthy organs of the subject, associated with clearance, such as the liver and kidney. Generally the removal of one or more of the above species is completed 15 minutes to 48 hours prior to the administration of the TAB-VL or TAB-TL. It should be realized that the Targeting Ligand may be a species that is not an antibody species and its binding to the targeted site in the organism, may be based on non-immunologic binding, as detailed and described in the incorporated references, in particular U.S. Pat. Nos. 6,039,946 and 5,753,227 and their counterparts and in Pimm, supra, Gurkavich, supra and Goodwin, supra. Thus the targeting ligand may be a ligand that binds non-immunologically to a receptor such as epidermal growth factor receptor, or it may be the peptide hormone somatostatin, that binds to the somatostatin receptor, which is present in high concentration in many tumors. The targeting ligand can also be other targeting proteins or peptides (OTP), or they can be non-peptide targeting molecules such as a drug, for example Atenolol that binds to beta-adrenergic receptor and Haloperidol, that binds the dopamine D2 receptor.

My U.S. patents and patent applications supra and in particular PCT applications WO 96/37516 provide for improved targeting over the previous art, by including a step of ECA of a species comprising circulating tumor antigen circulating tumor antigen-antibody complex and circulating antibodies, for example, enhancing antibodies, specific to tumor antigen.

These ECA methods will not be associated with competition of the administered unlabeled antibodies for the binding of the later labeled antibodies to the tumor antigen at tumor sites. These methods will also not have the risks and the undesirable effects of administering of unlabeled antibody to the organism, (infection, reaction to foreign protein immune complex disease). Optionally the methods of my patents supra can also add a step of removing of the targeted treatment or diagnostic ligand by extracorporeal affinity adsorption at a predetermined time following the administration of the treatment ligand or visualization ligand to further improve targeting. ECA removal of targeted ligands is disclosed in U.S. Pat. Nos. 6,046,225 and 6,251,394 and PCT application WO 96/37516.

The removal of circulating immune complexes can be achieved in accordance with my patents supra by specific extracorporeal immunoadsorbents such as anticomplex antibodies, or by Protein A affinity adsorbents (Fresenius Immunosorba®), Fresenius Prosorba®, Fresenius C1q extracorporeal adsorbent Miro®, Kaneka Selesorb® as well as Asahi, as disclosed in U.S. Pat. No. 4,627,915, as examples. Enhancing tumor antibodies can be removed for example, using affinity adsorbents such as tumor antigen, including synthetic, including analogs of antigen binding sites of tumor antigen, Protein A Immunosorba®, Prosorba®, for example, C1q, (Miro®, for example) as well as use of the adsorbents as used in Selesorb® and the above Asahi patent. Removal of circulating free tumor antigen (not complexed with antibody) can be achieved by the use of adsorbents such as specific antibody to tumor antigen.

It will be realized that tumor targeting inhibitor factors TIF may exist in complexes containing more than two molecular species. Such complexes may be affinity labeled for adsorption or adsorbed in the ECA column, by an affinity label or affinity adsorbent specific to any of the components of the complex, or specific to epitopes that are specific to the complex. Examples of such complexes are: CA-TAB-ATAA and NAB-CA-TAB. For example, the complex CA-TAB-ATAA may be adsorbed by antibody to any of its three components, bound non-covalently (e.g., by ligand) to Protein A that is bound covalently to the matrix in the ECA column, or it may be adsorbed by biotinylated antibody that is bound to Avidin in the Avidin ECA column. Similarly, the CA-TAB-ATAA can be affinity labeled for adsorption, by administering to the subject a biotinylated antibody, specific to any of its three components, to enable its specific adsorption in the ECA Avidin column or affinity labeled by antibodies to any of the components of the complex (or antibodies to epitopes specific to the complex) and adsorbed by ECA on a Protein A column.

In accordance with the current invention novel devices and methods are provided for the extracorporeal adsorption and removal of molecular and/or cellular tumor immunity suppressor factors (TISF), such as: TGFβ, p15E and Sialomucin, suppressor T cells (K. E. Hellstrom and I. Hellstrom, Encyclopedia Of Immunology, I. M. Roitt and P. J. Delves Eds., Academic Press 1992, pp. 1530-1531.), soluble receptor for tumor necrosis factor alpha (s R TNF alpha) and soluble receptor for tumor necrosis factor beta (s R TNF beta), soluble receptors for interleukins 1, 2, and 6 (sR IL-1, sR IL-2, sR IL-6) and soluble receptor for gamma interferon (s R INF-gamma). (M. R. Lentz, Therapeutic Apheresis Vol 3 (1) p 40-49, 1999 and M. R. Lentz, U.S. Pat. No. 6,231,536 B1. U.S. Pat. No. 6,231,536 provides for extracorporeal adsorption of soluble cytokine receptors by affinity ligands bound to matrix, including the optional addition of treatment with untargeted anticancer drugs. The current invention provides for extracorporeal adsorption of both soluble cytokine receptors as well as other molecular tumor blocking factors and suppressor cells. In accordance with some elements of the current invention the binding of the affinity adsorbent to the matrix in the extracorporeal device is done by binding the adsorbent, when the adsorbent is an antibody to the adsorbed (removed) species, to Protein A that is covalently bound to the matrix of the extracorporeal device, through the Fc part of the antibody, or by binding the affinity ligand, such as Avidin, covalently to the matrix and binding the adsorbent covalently to an affinity counterpart ligand (such as binding Biotin to the antibody adsorbent or to the cytokine adsorbent). The affinity counterpart ligand (Biotin, for example) covalently bound to the adsorbent (adsorbent antibody, for example) is then bound to the covalently matrix-bound Avidin, by non-covalent (by ligand) binding of the Biotin in the biotinylated antibody. The advantage of using Protein A column bound to the adsorbent antibody via the Fc part (domain) of the antibody include: ease of preparation and correct orientation of the adsorbent antibody antigen binding site, for maximal interaction with the antigens (Ed Harlow and David Lane: “Antibodies, A Laboratory Manual, pp: 518-521, Cold Spring Harbor Laboratory Pub, 1980).

The current invention also provides for the optional use of targeted anticancer treatment ligands and of visualization ligands, including optional use of targeting improvement by extracorporeal adsorption of targeting-inhibitors prior to the administration of the targeted ligand and/or post administration extracorporeal adsorption of the targeted ligand, after its administration. With respect to the treatment of AIS, the improvement that is the subject matter of the current invention is particularly aimed at providing extracorporeal adsorption devices and methods for the effective removal and reduction of MIFs and CIFs (together: MCIFs) species that are etiological in the pathogenesis of AS in particular AIS and most particularly in UA.

It is well known that coronary artery disease (CAD) and in particular its acute ischemic syndromes (AIS) are a major cause of morbidity and mortality. Revascularization procedures (coronary balloon angioplasty, angioplasty with stent implantation and coronary bypass surgery) are used when clinically indicated during AIS even though the associated morbidity and mortality is increased. Improved treatment of AIS, in particular UA, is highly desirable and acutely needed, in particular a treatment that can reverse some of the pathological processes in the atheromas, thus enabling to stabilize the disease process, so that mortality from the disease episode is reduced and the patient can either improve without surgical intervention, or have sufficient stabilization to be able to undergo by-pass surgery or angioplasty, with reduced risk of morbidity and mortality associated with such procedures when they are performed without achieving sufficient level of stabilization of the pathological changes in the atheroma sites and as a result, not achieving sufficient level of stabilization in the patient's clinical condition. While lipid reducing medications can be associated with some regression in coronary atheromas, when used long term, they have little use in the setting of AIS. Extracorporeal Affinity and Extracorporeal Filtration methods aimed primarily at reduction of Low Density lipoproteins (LDL), while having a more pronounced and more immediate effect on atheroma pathology may still have limited use in AIS.

Extracorporeal affinity adsorption methods aimed at removing from blood and body stores additional pathogenic chemical species, such as oxidized LDL and other pathogenic chemical species as well as pathogenic cellular species may provide the needed non-surgical treatment that will meet the goal of inhibiting pathogenic processes in atheromas, resulting in sufficient quantitative changes within a short time frame, to enable pathological and clinical stabilization of patients with AIS, in particular UA, so as to either avoid the need for surgical or other invasive intervention, or alternatively, enable pathological and clinical stabilization, so that invasive revascularization procedures can be used with reduced morbidity and mortality.

The following published documents are incorporated herein by reference in the current application in their totality: U.S. Pat. No. 6,039,946, U.S. Pat. No. 5,753,227, and their parent application U.S. Ser. No. 97,378, filed Jul. 23, 1993 as well as its corresponding PCT application published as WO 95/03084. The references cited therein are also incorporated by reference herein.

Numerous molecular inflammatory factors (MIF) and cellular inflammatory factors (CIF) have been identified as etiological in the pathogenesis of atherosclerosis (AS), including coronary artery disease (CAD), peripheral atherosclerotic vascular disease (PASVD), atherosclerotic cerebro vascular disease (ASCVD). Included in CAD are acute ischemic syndromes (AIS) that include acute myocardial infarction (MI) and unstable angina (UA). MIF combined with CIF are referred to in the application as “Molecular and Cellular Inflammatory Factors” (MCIF).

The MCIFs involved in the etiology and pathogenesis of atherosclerosis (AS) and in particular UA, including those that are increased systematically, are summarized by V. Pasceiri and E. T. H. Yeh, Circulation 1999, Vol. 100, pp. 2124-2126. The molecular MCIF species increased in UA include Interleukin 6 (IL-6), IL-2 Receptor, C Reactive Protein (CRP) and TNF alpha. Cellular MCIFs include: CD3+ DR+ lymphocytes, CD4+ C28-(null) T cells, CD4+ NF gamma+ T cells, Th1 T cells.

Publications reporting the involvement of inflammation processes in AS and in particular AIS include: Z. Reiner et al., Lijec. Vjesn. 2001, January-February, Vol. 123 (1-2) pp. 26-31. They found that Interleukin 2 (IL-2) was increased in UA and the level of IL 2 was correlated with the level of serum lipids. Increased levels of metalloproteinases and proteolytic enzymes may also be associated with UA. Koukkunen et al., Annals. Medicine, Vol. 33 (1) pp. 37-47 reported increased C Reactive Protein (CRP), IL-6, TNF alpha, fibrinogen, Troponin and creatine kinase in UA. K. K. Mizzia-Stec et al., Pol Arch med Wewn 1999, August; Vol. 102(2), pp. 677-84, reported that both TNF alpha pro-inflammatory cytokines and IL-10, anti-inflammatory cytokines, were increased in both stable and unstable angina. A. D. Simon et al., J. Thrombosis and Thrombolysis, 2000, April, Vol. 9. (3) pp. 217-22, found increased IL1-beta and IL-6 in UA. W. H. Lee et al., Exp. Mol. Med. Sep. 30, 1999, Vol. 31(3) pp. 59-64, found increased level of CRP in plasma of patients with UA. M. Biasucci et al., Circulation, 1999, Apr. 27, Vol. 99 (16) pp. 2079-84, found that IL-6 and IL-1Ra were elevated in UA and that their level correlated with the likelihood of a complicated hospital course. G. Caligiuri et al., Circulation, 2000 Sep. 5, Vol. 102 (10) pp. 1114-9, found that oxidized LDL (OX-LDL) is an antigen that causes the specific proliferation of T cells of patients with UA, thus indicating that OX-LDL is involved in the etiology and pathogenesis (EP) of UA. A. Mazzone et al., Atherosclerosis, 1999, August, Vol. 145(2) pp. 369-74, found increase of IL-6 in Ac al., W. H. Lee et al., Exp. Mol. Med., 1999, Sep. 30, Vol. 31 930, pp. 159-64 found elevated CRP in MI and UA. M Hoffmeister et al., Am J. Cardiol., reported elevated CRP in AIS. D. A. Smith et al., Circulation 2001, Aug. 14, Vol. 104 (7) pp. 746-9, found that IL-10 has a protective role in AS. N. Nakajima et. al., Circulation 2001, Feb. 5, Vol. 105 (5), pp. 570-75, reported elevated CRP in UA. K. A. Fox et al., Brit Med Bull, 2001, Vol. 59, pp 69-87, pointed out that management of associated risk factors, such as hypercholesterolemia appears to have substantial benefits even during the acute in-hospital phase of Acute Angina. E. Lindmark et al., JAMA 2001, Nov. 7, Vol. 286 (17), pp. 2107-13, reported elevated IL-6, CRP and Fibrinogen in UA, particularly a strong independent effect of elevated IL-6 in association with increased mortality in UA.

J. Plutzky, Am. J. Cardiol., 2001, Oct. 18, Vol. 88 (8A), pp. 10K-15K, reported elevated CRP, TNF alpha and IL-6 in AIS. Bayes-Genis et. al., New England J. Med, 2001, Oct. 4, Vol. 345 (14), pp. 1057-9, reported increased level of pregnancy associated plasma Protein A (PAPP-A) in the plasma and in unstable plaques in AIS.

H. Lee et al., Int. J. Cardiol., 2001, September-October, Vol. 80 (2-3), pp. 135-42, reported increase in CD14 monocytes and HLA-DR+ T lymphocytes in the acute phase of CAD and specifically reported the CD14+ expression on monocytes and percentage of HLA-DR+ T cells are decreased during treatment and that the expression of CD14 represents the activation of monocytes during the acute phase of CAD. W. H. Lee et al., Cardiology, 1999, Vol. 92 (1), pp. 11-6, reported data indicating that the rupture of an atherosclerotic plaque and formation of thrombus may lead to activation of CD40 cells in platelets of patients with AIS.

G. Liuzzo et al., Circulation, 1999 Nov. 23, Vol. 100 (21), pp. 2135-9, reported an increase in number of CD4+ CD28null T cells in UA. As discussed in Pasceri et al., supra, this increase in T cell population is associated with increased production of INF gamma, that stimulates macrophages to produce metalloproteinases and other proteinase enzymes. P. Aukrust et al., Circulation, 1999, Aug. 10, vol 100 (6), pp. 614-20, reported that T cells positive for the CD40L soluble and membrane bound ligand on activated T cells and platelets, particularly in UA, may play a role in the triggering and maintenance of AIS. CD40L cells induced enhanced release of chemoattractant peptide from monocytes, a chemokine involved in the pathogenesis of AS. T. Nakajima et al., Circulation 2002, Feb. 5, Vol. 105(5), pp. 570-75, reported increased level of CD4+ CD28null (null) T cells in AIS. These cytotoxic cells were reported to efficiently kill endothelial cells in vitro and the killing is increased by sensitizing the target cells by CRP. The authors found increased frequency of Perforin and CD16 expressing CD4+ T cells in the peripheral blood, as well as increased CD161 appearance on null cells. Perforin expressing CD4+ cells from UA patients were cytotoxic to endothelial cells In Vitro.

A. Mazzone et al. Atherosclerosis, 1999, August, Vol. 145(2), pp. 369-74 found increased IL-6 in AIS and also found that IL-10 had a protective role in AS. D. A. Smith et al., Circulation, 2001, Aug. 14, Vol. 104 (7), pp. 746-9, reported increased IL-10 in stable angina and reduced IL-10 in UA, indicating a protective effect of II-10 in AIS.

S. Yazdani et al., American Heart J., 1998 August, Vol. 136 (2) pp. 357-61, found data indicating that increased IL-6 is correlated with destabilization of atheroma in coronary arteries. H. Tashiro et al., Coronary Artery Disease, 1997 March, Vol. 8 (3-4), pp. 143-147, found that Macrophage Stimulating Factor MCSF is stimulating and Transforming Growth Factor Beta is (TGF-Beta) is inhibiting the process of AS. D. A. Smith et al., Circulation, 2001, Aug. 14, Vol. 104 (7) pp. 746-9, reported protective effect of IL-10 in AS.

G. Luizo et al, Circulation, 1999, Nov. 23, Vol. 100(21) pp. 2135-9 reported in UA increase in CD4+ CD28null cells associated with INF gamma production that was associated with persistent antigenic stimuli. D. E. Newby and K. A. Fox Br Med Bull 2001, Vol. 59, pp. 69-87, reported that use of anti-inflammatory clopidogrel and glycoprotein IIb/IIIa receptor antagonists had positive effect on UA.

The improvement that is the subject matter of the current invention is particularly aimed at providing extracorporeal adsorption devices and methods for the effective removal and reduction of MIFs and CIFs (together: MCIFs) species that are etiological in the pathogenesis of AS in particular AIS and most particularly in UA. As mentioned in the background section, the data indicating the implication of MCIFs in etiology and pathogenesis (EP) of AS and AIS are present not only in AS but also in other inflammatory and autoimmune disease (such as rheumatoid arthritis) and may be implicated in non-atherosclerotic vascular diseases such as those associated with autoimmune disease, thus the devices and the methods of the present invention may be utilized in treatment of these diseases as well as in cerebro vascular disease and peripheral vascular disease. Utilization of the methods and devices can also be applicable to inflammatory diseases in general, including inflammation associated with infectious and some neoplastic diseases. M CIFs can be reduced not only by ECA affinity adsorption but also by increased metabolism and clearance, which can be achieved by passive immunization with specific antibodies and including the use of catalytic antibodies (C Tellier, Transfus. Clin. Biol., 2002 Jan., Vol. 9, pp. 1-8) including fragments, including synthetic fragments and analogs. Active immunization with immunogenic preparations of MIFs may have utilization in the treatment of more chronic aspects of AS, even though they would not have a role in the treatment of AIS. The MCIFs that are increased in AIS include the following: TNF alpha, metalloproteinases, IL-6, soluble IL2 receptors, CD3+ DR+ T cells, CD4+ CD28null T cells, CRP, INF gamma. Utilizing known methods for affinity removal of cells or using ECA affinity adsorption with specific adsorbents of MCIFs such as Mabs, including fragments, or other affinity ligands, such as receptors and synthetic receptors including synthetic receptor fragments and analogs, will lead to removal of MCIFs from the various compartments of the body including biological fluid compartments, such as blood, peritoneal and CSF for example, as well as and in particular from atherosclerotic plaques. The MCIF removal treatment may be combined with drug treatment that is currently used in AIS such as anti-inflammatory and fibrinolytic drugs, for example.

Autoantibodies specific to the β1 Adrenergic receptor have been implicated in the etiology and pathogenesis of Idiopathic Dilated Cardiac Myopathy (IDCM) (G Wallukat et al: New England J. Med. Vol 347, No 22, Nov. 28, 2002) who found that specific removal of these antibodies by the specific synthetic antigen in extra corporeal adsorption (ECA) leads to significant improvement in cardiac performance (utilizing Affina Coraffin® column). Numerous other autoantibodies have also been found in IDCM (Muller et al: Circulation, 2000, Vol 101, pp 385-391.) Of particular interest is the association of ICM autoantibodies specific to Oxidized LDL (I Schimke et al: J Am College of Cardiol, 2001, Vol. 38, No 1, pp 178-183) found that following ECA using a sheep anti-human-immunoglobulin antibody, as the adsorbent, the level of autoantibodies specific to oxidized LDL, correlated negatively with lipid peroxide levels (indicator of oxidative stress) as well as with left ventricular ejection fraction. ECA was associated with significant decrease in the level of autoantibodies to the β1 adrenergic receptor. P A Sobotka et al., Free Radical Biology and Med, Vol. 14, pp 643-647, 1993, reported reduced breath Pentane in Heart Failure, by the use of free radical scavengers (Pentane is produced as a by-product of lipid peroxidation). The use of the above general non-specific adsorbents (“non-specific”, with respect to adsorbing specific antibodies), namely: the use, for example, of sheep anti human immunoglobulin antibody, as the adsorbent for the removal (adsorption) of auto antibodies to the β1 adrenergic receptor in the treatment of IDCM, will remove many of the autoantibodies that contribute to the etiology and/or pathogenesis of the disease (autoantibodies to: β1 adrenergic receptor, oxidized LDL, ADP-ATP carrier, alpha and beta cardiac myosin heavy chain isoform, G protein coupled receptors, heart mitochondria).

However, using a non-specific adsorbent has the disadvantage of requiring the administration of replacement human Immunoglobulin, with the associated risks.

In accordance with the inventive improvement of the current invention, ECA treatment of IDCM uses as the adsorbent, more than one specific adsorbent, specific to the more than one of the above listed autoantibodies. The specific adsorbents used in the ECA may include the relevant antigens (for example: oxidized LDL, ADP-ATP carrier and the other antigens listed above), including in the adsorbents are synthetic antigens, including fragments and including analogs of the antigens, as well as antiidiotypic antibodies to the specific autoantibodies implicated in the etiology and pathogenesis of IDCM. In addition, in accordance with the current invention, the ECA may include as an adsorbent a chelating agent, such as EDTA, for example, to adsorb oxidants such as iron and copper, for example, in order to reduce oxidative stress.

The devices of the present invention make possible the use of a single device having contained therein a binding compound bound to a carrier, the binding compound having affinity for a binding partner, which may later be combined by a user with one or more of a plurality of affinity binders, each of the affinity binders comprising a first portion comprising the binding partner and a second portion adapted to bind selectively with a species, the second portions of each of said affinity binders differing from each other. The generic columns can be produced and shipped without the affinity binders, and the user can easily select and bind the affinity binder to the column by passing a solution of the affinity binder through the column before it is used. Alternatively, the affinity binder or binders can be bound to the generic column prior to shipment. The binding partners are illustratively Avidin and Biotin, permitting the use of a single, possibly certified, Avidin column to produce columns which bind selectively to a vast number of species which are naturally present in the treated mammal, or are introduced into the mammal being treated, such as by administering the species (such as a drug) to the mammal being treated, or by self-overdose of a prescription or over-the-counter drug or of a drug of abuse, or by self administration, accidental or deliberate, of a toxic chemical substance such as a poison. These species may also include, for example, TNF alpha, IL-6, CD3+ DR+ T cells, CD4+ CD28null T cells, CD3+, CD56+, DM1, VGO1, LAK1, CRP, INF gamma, CA, NAB, CA-NAB, TGF β, P15E, Sialomucin, TH2 T cell epitope, tumor infiltrating lymphocyte (TIL) marker, lymphokine activated killer cell (LAK) marker, Interleukin 10 (IL-10), prostaglandin E2 (PGE2), mucin, suppressive E receptor (SER), immunosuppressive acidic protein (LAP), adhesion molecules, sR TNF alpha, sR TNF beta, sR IL-1, sR IL-2, sR IL-6, sR INF gamma, heat shock protein (HSP), antibodies to oxidized LDL (Ab-OxLDL), antibodies to HSP, CRP, triglycerides, IL-2, metalloproteinases, other proteinases, fibrinogen, creatine kinase, IL-1-Beta, IL-I-Ra, PDGF, angiotensin II, MCSF, pregnancy associated plasma protein A (PAPPA), antibodies specific to any of the following: the β adrenergic receptor, ADP-ATP carrier, alpha cardiac myosin heavy chain isoform, beta cardiac myosin heavy chain isoform, G Protein coupled receptors, and heart mitochondria, and toxins selected from the group consisting of botulinum toxin, tetanus toxin, ricin toxin, ricin A peptide toxin, sulfur mustard toxin, and toxic metabolites thereof.

The devices of the present invention may also be used for other purposes. For example, as extracorporeal devices, they may be used for collecting and analyzing species in mammalian fluids. They may also be used other than for extracorporeal removal of species from mammals. For example, they may be used for removal of chemicals or cellular species from non-biological fluids, for example to purify the fluid by removing a species or to collect the species. For example, the species may be used for therapeutic purposes, or to identify it as a contaminant in the fluid.

When affinity adsorption is used, in accordance with the present invention, optionally the affinity adsorbent can be regenerated by the removal of the adsorbed species, in particular when the adsorbed species is a chemical species, thus enabling the continuous use of the adsorbent for further adsorption of the species. Numerous methods for species elution from the affinity adsorbent can be utilized as disclosed in E. Harlow and D. Lane, “Antibodies a Laboratory Manual,” Cold Spring Harbor Laboratory Pub., 1988, pp 511-552, and in particular p. 547-552. Elution methods that enable adsorbent reuse are utilized in accordance with the invention. Examples of these methods are listed in Table 13.4 p. 549) of Harlow and Lane, supra, and include high pH buffer, for example triethylamine, pH 11.5; low pH buffer, for example 100 mM glycine buffer, pH 2.5; high salt solution, for example 5M LiCl, 10 mM phosphate, pH 7.2; dissociating agents, for example 2M urea; and organic solvents, for example 50% ethylene glycol pH 8.

### BRIEF DESCRIPTION OF DRAWINGS

FIG. 1 is a diagrammatic view, showing one illustrative embodiment of a device embodying one aspect of the present invention for carrying out a method of the invention.

FIG. 2 is a diagrammatic detail of the device of FIG. 1, showing a binding compound bound to a carrier in the device, the binding compound being bound by affinity binding to a binding partner.

### BEST MODE FOR CARRYING OUT THE INVENTION

The following Examples are given merely by way of illustration of the invention, and are not meant to be limiting.

### EXAMPLE 1

### Step One of Extracorporeal Removal of CA-NAB, and/or NAB and/or ATAA Prior to Administration of TAB-VL or TAB-TL

Protein A-Sepharose® CL-4B obtained from Pharmacia LKB Biotechnology and swollen and washed in accordance with the instruction (Affinity Chromatography. Principles and Methods, Pharmacia Biotechnology Pub., 1991), is packed in a column. Preferably the column used is the commercially available Immunosorba®) sold by Fresenius HemoCare Inc. Redmond, Wash. The column requires the use of a plasma separator, as is well known and as recommended by the manufacturer: either a “centrifuge” type, such as Fresenius AS 104 cell separator, Cobe IBM 2997 or membrane type plasma separator, such as Kaneka Sulfox® or Cobe TPE® can be used to separate, on line, the patient's plasma from the cellular elements of blood. While the Immunosorba® column is preferred in some applications, other Protein A Adsorption columns can be used instead. When regeneration is preferred a system including two Immunosorba® columns with a Fresenius Automatic Regeneration unit, Citem 10® are used. When no regeneration of the adsorbent column is needed only a single Immunosorba® column can be used. When it is preferable to use a disposable column, the column utilized for adsorption can be the Fresenius HemoCare Prosorba® column in which the Protein A is covalently bound to a silica matrix. It should be realized that other Protein A columns can be used instead, using various biocompatible matrixes to which the Protein A is covalently bound. (Whenever Protein A is mentioned herein the term includes, peptide fragments of Protein A, including synthetic peptides, that are all known in the art to be used as equivalents of Protein A.) The matrixes used include natural polymers, such as cellulose and dextran, various synthetic polymers and copolymers, such as polyacrylamide, polystyrene and polyvinyl polystyrene copolymer, and silica and silicones. One suitable matrix is heparinized silicone described in D. R. Bennett et al., U.S. Pat. No. 3,453,194.

The configuration of the matrix is also not limited to a particular form, examples of suitable forms are beads (in particular, spherical in shape), fibrous matrixes, macroporous matrixes and membranes, including hollow fibers. One possible device is that of a typical multi-hollow fiber filtration, dialysis or diafiltration design. Such a configuration consists of a bundle of hollow fibers encased in a tubular housing. The adsorbent is included in the outer space of the hollow fiber, as disclosed in my U.S. Pat. No. 6,039,946, see in particular column 9, lines 9-38 and FIGS. 2 and 3; the patent uses in the example a chelator as the adsorbent, but in the current invention the adsorbent is Protein A, either in free form, bound to a matrix, particularly by covalent chemical binding. or alternatively the Protein A can be bound to the membrane, to its blood flow side to the side opposite to the blood flow or to both sides, rather then being incorporated in the outer space of the hollow fiber. The Protein A, in either free form, or matrix bound, can also be physically trapped in the membrane, (such as when the membrane is an unisotropic polysulfone membrane, for example the one produced by Amicon). When the Protein A is encapsulated it can be encapsulated in one of the microcapsules or macrocapsules as described in U.S. Pat. No. 5,753,227, in particular column 7, line 53-65, or the double encapsulation described by Markus et al., American Heart Journal, Vol. 110 No 1, part 1, July 1985, pp. 30-39. In the practice of the invention adsorbents other than Protein A, or in some situations, in addition to Protein A, can be used, that can adsorb CA, CA-NAB, NAB or ATAA. These adsorbents include for example, TAB (that will bind CA, CA2-NAB and ATAA) Protein G, C1q bound to antiC1q antibody, covalently bound to matrix, C1q covalently bound to matrix (for example, Miro® sold by Fresenius HemoCare) Dextran Sulfate (for example, Selsorb® sold by Kaneka Corp.) and the adsorbent disclosed in Kuroda et al., U.S. Pat. No. 4,627,915. Optionally, the Protein A extracorporeal column adsorbent can incorporate specific adsorbents, as detailed in U.S. Pat. No. 5,753,227, column 10, line 51 to column 11, line 9. In the application of this method in accordance with the current invention an intact antibody, or an antibody fragment containing Fc fragment, and specific to a molecular species, desired to be removed, in accordance with the current invention, is bound to the protein A in the column by none covalent binding, following procedures known to those skilled in the art (see for example, E. Harlow and D Lane, Antibodies, A Laboratory Manual, Cold Spring Harbor Laboratory Pub., pp. 511-527, 1988, and Affinity Chromatography, Principles and Methods, Pharmacia LKB Biotechnology Pub. # 18-1022-29, pp. 47-52). In accordance with the present invention the antibodies bound to the protein A in the ECA column are one or more of the following specific antibodies (SA): Antibody to TAB, antibody to CA (for example TAB) antibody to NAB, (anti idiotypic antibody) (antibody to ATAA (e.g. anti idiotypic antibody), CA-NAB, NAB, antibody to tumor blocking and tumor immunity suppressor factors and suppressor cells such as: TGF β, p15E, Sialomucin, sR TNF alpha, sR TNF beta, sR IL-1, sR IL-2, sR IL-6, sR INF gamma, TH2 T cell epitope. The specific antibodies coupled to Protein A are crossed linked to the Protein A by a bifunctional coupling reagent, preferably dimethylpimelimidate (DMP), or by another coupling reagent, in accordance to Harlow and Lane Supra. In the Example, the organism being treated is a human, the adsorption system used is the Fresenius system containing two Immunosorba® columns and a Citem 10® regeneration unit. Each column contains 62.5 ml of Protein A, which is covalently bound to a cross-linked beaded Sepharose matrix. Plasma is first separated from blood cellular elements, using Fresenius AS 104 Cell Separator. To the Protein A in the column, are bound, through the Fc binding site of the Protein A, intact antibodies to at least one of the above specified chemical or cellular species (TAB, CA, NAB (anti idiotypic antibody), ATAA (anti idiotypic or anti isotypic antibody), CA-NAB, TGF β, P15 E, sialomucin, sR TNF alpha, sR TNF beta, sR IL h1, sR II 2, sR IL6, sR INF gamma, TH2 T cell epitope. In the EXAMPLE 1, the matrix bound Protein A is bound to the Fc part of antibody ZCE-025 specific to CEA antigen (to adsorb circulating CEA and to adsorb ATAA that is specific to antibody ZCE-025. Mab CH-255 is bound via Fc to Protein A in the ECA column to adsorb the VL L-SCN—C₆H₄—CH2-EDTA-In.

In Step 1 of EXAMPLE 1, the plasma flow rate in the Immunosorba® column is approximately 20-35 ml/min. Flow rates can vary, depending on the individual case and can be in a range from 5 to 50 ml/min, when Immunosorba® column is used.

Depending on the individual case and the particular column used, persons with skill in the art can determine without undue experimentation the appropriate flow rate, in the individual case. Step 1 above can be completed preferably from 0.1 hours to 24 hours prior to Step 2 of TAB-VL or TAB-TL administration. Most preferably it will be completed between 0.15 hours to 4 hours, prior to TAB-VL or TAB-TL administration. The length of Step 1 is typically between 1 hour and 17 hours and more typically between 2 hours and 4 hours, depending on the individual case including, for example, age, weight, presence of pathological fluids in body cavities, such as peritoneum, pericardium and pleural cavity, as examples. It should be understood that while in the EXAMPLE, plasma is being treated, other bodily fluids can be treated, such as peritoneal fluid, lymph, cerebro spinal fluid, with access to these biological fluids achieved by methods that are well known in the art. When fluid of the blood circulatory system is accessed and treated, the fluid may be blood rather then plasma (see for example U.S. Pat. No. 5,753,227, including use of encapsulated adsorbent, when blood is directly treated). Following the adsorption Step 1, in the EXAMPLE, in Step 2, the TAB is administered intravenously. The TAB in the EXAMPLE is a TAB-VL, which is a hybrid of the two intact monoclonal antibodies Mab CHA-255, specific to the hapten L-SCN—C₆H₄—CH2-EDTA and Mab ZCE-025 specific to CEA (C. Lollo et al., Nuclear Medicine Communications, Vol. 15, pp. 483-491, 1994). The VL is ¹¹¹In-NBE-EDTA, which is bound non-covalently to the antibody binding site of Mab CHA-255. The hybrid of Mab CHA-255 and Mab ZCE-025 is prepared by using the method described by Lollo et al. supra, except that instead of the hybrid F(ab′)2, a hybrid of the intact antibodies (which is the preferred species used) is the hybrid utilized. Alternatively it is possible to use hybrid of one intact Mab with the Fab fragment of the second Mab thus including in the hybrid an Fc piece of at least one antibody molecule, to enable binding to Protein A and Protein G. used as the adsorbent in the extracorporeal column.

In Step 3 of the current EXAMPLE 1, following the administration of the Fc containing hybrid (such as the hybrid antibody of Mab CHA-255 and Mab ZCE-025), the adsorbent in the ECA column may be Protein A, that will adsorb from the treated fluid (such as plasma or blood, for example) the hybrid antibody, including hybrid antibody bound to the VL 111-In-NBE-EDTA that was not targeted to the tumor site and continued to circulate in the blood (or other biological fluid).

As described above, the adsorbent used in the ECA column includes Mab CHA-255 bound to Protein A or Protein G. through Fc of the Mab.

Mab CHA-255 will adsorb NBE-EDTA that was not targeted to the tumor site and continued to circulate in the blood (or other biological fluid). Whenever an antibody or Fc containing antibody fragment is bound to Protein A, it can be, optionally cross linked to the Protein A via a bifunctional coupling agent such as dimethylpimelimidate (DMP) (Harlow and Lane, supra, pp. 521-527).

The adsorbent in the ECA column can also be EDTA that is covalently bound to the matrix, preferably through a spacer arm having a length of between 5-20 carbon atoms. Alternatively the EDTA can be conjugated to IgG that will bind to the Protein A through The IgG Fc. The EDTA will adsorb free 111-In, released in the blood circulatory system (or released to other biological fluid, being treated in the ECA column). Step 3 will preferably start 30 minutes to 72 hours after completion of Step 2, most preferably it will start 2-48 hours after completion of Step 2. Step 3 will preferably last 2 to 48 hours, most preferably 2 to 24 hours.

A modification of the method is to use liposome incorporation of VL in accordance with WO 96/37516. The incorporated ligand may be TAB-VL (for example, Mab ZCE-025-Mab CHA-255NBE-EDTA-111-In) or it may be any other ¹¹¹In containing species, such as EDTA-111-In and target the liposome to the tumor, by binding to the wall of the liposome, covalently, or by ligand (non-covalently) Mab ZCE-025, or its fragment that will target the liposome to the tumor. Instead of a Protein A ECA column, a Protein G ECA column can be used.

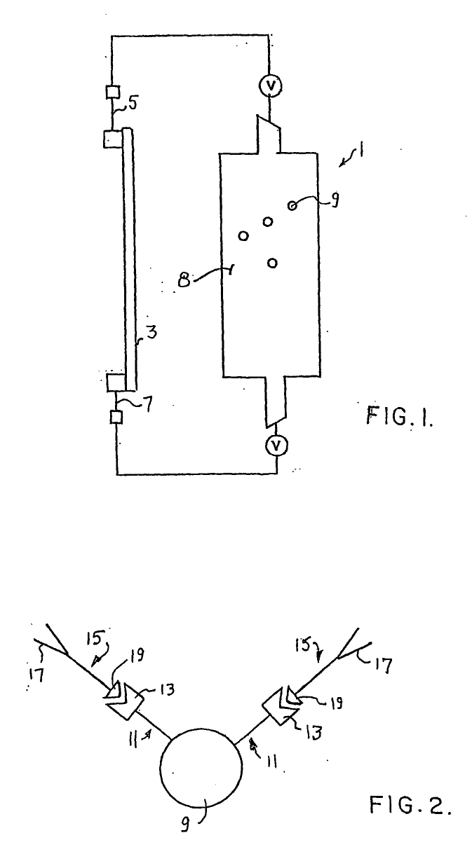

Alternatively to using a cross-linking bifunctional (or multifunctional) coupling reagent in accordance with Harlow and Lane cited supra, to cross link the adsorbing antibodies to the Protein A bound to the matrix in the column, the ECA can be used without cross linking (coupling) the adsorbing antibodies to the Protein A, by the use of a cross linking reagent. In this optional configuration, when regeneration of the adsorbing antibodies is desired, this can be achieved, in extracorporeal adsorption column (FIG. 7 of U.S. Pat. Nos. 4,375,414 and 4,813,924 and FIGS. 4, 5 and 6 of U.S. Pat. Nos. 5,753,227 and 6,039,946). Following the step of adsorption of the adsorbed species by the adsorbing antibody (or other Fc containing adsorbent), which is bound, by ligand (non-covalently) to the Protein A (or Protein G) that is bound covalently to the matrix in the column. The ECA column is regenerated by regenerating means, preferably, regenerating fluid, E. Harlow and D Lane; Antibodies A Laboratory Manual, pp. 547-552, Cold Spring Harbor Laboratory Pub. 1988. Preferably, the eluting fluid is an acidic buffer, such as for example glycine phosphate buffer pH 2.5-3; such treatment will split both the adsorbing antibody from the matrix covalently bound Protein A (or Protein G) and will also split the adsorbing antibody (or other Fc containing adsorbent) from the adsorbed species. The acidic eluting buffer solution is then transferred to another column that contains an adsorbent that will specifically bind the adsorbing antibody, but will not bind the adsorbed species, when the pH of the buffer solution is changed to a pH in which binding takes place such as by adjusting, for example the pH to 7.4 by addition of a base, such as for example NaOH, with stirring, by methods known in the art, such as use of a magnetic stirrer, or vibration, for example. Preferably, the removal of the adsorbed species is achieved by filtration of the acidic buffer through a semipermeable membrane, permeable to the buffer solution and to the adsorbed species but not permeable to the adsorbent antibody. Pumps are used, as needed for pressure filtration. A regeneration buffer such as PBS pH 7.4 is added to the adsorbent antibody in the regeneration column. Optionally, the adsorbent in this regeneration column is an antidiotypic antibody specific to the adsorbing antibody, and binds the adsorbing antibody through the antibody binding site of the adsorbing antibody, thus not enabling binding of the adsorbed species to the adsorbing antibody, or the adsorbent in the regeneration column is the adsorbed species bound to the matrix in the column. In the preferred regeneration method, using membrane filtration, the adsorbent may be an antiisotypic antibody. Prior to changing the pH of the solution to 7.4, the adsorbed species is removed from the regeneration column, for collection, chemical analysis or discarding. The adsorbing antibody is then eluted again from the adsorbent in the regeneration column, such as by use of acid pH buffer, returned to the ECA adsorption column, where the pH is again adjusted to 7.4, by adding of NaOH, or by filtering out the acidic buffer and the adsorbed species by membrane filtration and adding a regenerating (binding) buffer, such as PBS pH 7.4, for example. The ECA adsorbing column is then ready for continued use or later reuse, as in this pH the adsorbing antibody will again bind the Protein A or Protein G in the ECA column. Alternatively to membrane filtration, the adsorbent, such as the adsorbent antibody, can be separated from the adsorbed species by other known separation methods, such as centrifugation or gel filtration, for example. The same column can be used for regenerating the membrane based adsorbing devices and methods (for example FIG. 6 of U.S. Pat. Nos. 4,813,924 and 4,375,414 and FIGS. 1, 2, and 3 of U.S. Pat. Nos. 6,039,946 and 5,753,227), wherein the adsorbent is an antibody (or another Fc containing adsorbent) bound by ligand (non-covalently) to Protein A or Protein G, that is covalently bound to the matrix in the column. Alternatively to the use of a regeneration column as described above, the regeneration can be achieved by adding the elution solution (such as an acidic buffer) to the ECA column following the adsorption step of the adsorbed species to the antibody or other Fc containing adsorbent in the ECA column and separating the adsorbed species from the adsorbent antibody, following the step of elution, by known separation techniques, such as membrane filtration, gel filtration or centrifugation, for example, followed by returning the adsorbing antibody to the ECA column and removal of the adsorbed species.

### EXAMPLE 2

This Example is identical to EXAMPLE 1 above, except that the Step 3 of ECA of TAB-bound 111 In NB-EEDTA-111 In, or free ¹¹¹ In is omitted.

### EXAMPLE 3

This Example is identical to EXAMPLE 1, except that the ECA column is a Protein A (or Protein G) column and wherein no antibodies are used as adsorbents (the adsorbent is the Protein A or Protein G itself covalently bound to the matrix) and wherein the Adsorbed Species is a species which has affinity to Protein A (or Protein G), such as for example ATAA, NAB, TAB and any other Adsorbed Species that has affinity to Protein A or Protein G. IN this EXAMPLE 3, an additional Step can optionally be added, prior to Step 1 of ECA, in order to increase the affinity of the adsorbent Protein A, (or protein G) used in the EXAMPLE, and when the Adsorbed Species is ATAA, monoclonal antibodies specific to the ATAA, (anti idiotypic antibodies to the FV binding site of ATAA), are administered to the treated subject. The production of monoclonal anti idiotypic antibodies to ATAA, are well known to those skilled in the art, as ATAA is a complete antigen. While use of monoclonal antibodies, preferably, chimeric or humanized antibodies are preferred, polyclonal antibodies can be used instead. Instead of administering anti ATAA antibodies, cold TAB, e.g. TAB that is not bound to a VL or TL, can be administered, instead. In either case the production of ATAA-Anti-ATAA or ATAA-TAB complexes will increase their adsorption by Protein A, hence their effective removal by ECA. It is realized that should TAB be administered it will also inherently have affinity to and bind CA TA as well as CA2NAB.

When affinity labeling is used, any adsorbed species (STBR) can be removed, and more specifically all of the adsorbed species, specifically mentioned in this patent application, that are either antigens or haptens and antibodies to them are known, or can be produced by those skilled in the art. Hence, in accordance with the current invention, antibodies (or other antibody Fc containing antigen or hapten binding antibody fragments) can be administered to the subject being treated prior to ECA on a Protein A column, thus enabling adsorption of any and all of the adsorbed species disclosed herein to the Protein A (or Protein G) in the ECA column. Following the administration of the TAB-VL in Step 2, to the subject, optionally, an antibody to the TAB-VL (such as an antiidiotypic antibody specific to the TAB component, an antiisotypic antibody specific to the TAB component, or an antibody specific to the VL component of the TAB-VL) can be administered to the subject being treated, followed by ECA using a Protein A (or Protein G) ECA column, in order to enhance the removal of the TAB-VL, following its administration. In the above applications of ECA, wherein the ECA step is used to remove complexes, C1q can be used as the adsorbent in the ECA instead of the Protein A or Protein G.

### EXAMPLE 4

This Example is identical to EXAMPLE 3 except that in order to enable the extracorporeal adsorption in Step 1 of free circulating tumor antigens (CA) that are not bound to NAB and therefore can not be adsorbed by the Protein A used as adsorbent (as well as Protein G or C1q. when these are used as adsorbents) as well as enable the more effective adsorption of ATAA, prior to step 1 of ECA, TAB (e.g. Antibodies specific to CA) is administered (intravenously, intraperitoneally or by other route, depending on the individual case). TAB will also bind to ATAA (as the Antigen of ATAA) producing ATAA-TAB complex, thus enhancing the adsorption of the ATAA by the Protein A Protein G or C1q adsorbent in the ECA column. The TAB antibodies are preferably monoclonal antibodies, preferably chimeric or humanized antibodies, but can be polyclonal antibodies. The amount of antibody administered can be determined by those skilled in the art, depending on the individual case and will generally be between 0.1 Mg/Kg to 2 mg/Kg, Preferably 1 mg/Kg. The time interval between completion of this antibody administration and Step 1, is relatively short, in order to reduce to minimum the access of unlabeled TAB to the tumor, thus reducing the amount of unlabelled TAB bound to TA, and reducing the competition of unlabelled TAB, with the Labeled TAB. In view of the fact that when unlabeled TAB is administered, such as by intravenous injection, it will have immediate access to CA in the BC, but a relatively delayed access to the tumor site, the time delay between completion of administration of unlabeled TAB and the initiation of Step 1 ECA, will generally be between 5 minutes and 6 hours, preferably between 10 minutes and 2 hours. The time will depend on the individual case and can be determined by those skilled in the art without undue experimentation.

### EXAMPLE 5

This EXAMPLE is similar to EXAMPLES 1-3, except that in order to enable adsorption of free NAB, by Protein A, anti idiotypic antibodies specific to NAB are administered prior to Step 1 of ECA.

### EXAMPLE 6

This EXAMPLE is similar to EXAMPLES 1-5, except that in order to enable adsorption of tumor immunity molecular suppressors, (Such as TGF beta, p 15E, sialomucin, sR TNF alpha, sR TNF beta, sR IL-1, sR IL-2, sR IL-6, sR INF gamma) or tumor immunity cellular suppressors (such as TH2 suppressor cells) (TISF), antibodies to the suppressor molecules and/or suppressor cells (e.g. in the example, antibodies to Th2 epitopes) is administered, preferably, prior to Step 1 ECA, or using an ECA wherein the antibodies to the TISF are bound to the matrix in the ECA column.

### EXAMPLES 7-12

These EXAMPLES are similar to examples 1-6 except that the targeted ligand is the TL adriamycin, which is administered in accordance to EXAMPLE 2 of WO 96/37516, incorporated herein by reference.

### EXAMPLES 13-19

These EXAMPLES are similar to Examples 7-12 except that the adriamycin is covalently bound to an intact Mab specific to the Tumor Antigen (TA) alpha-fetoprotein (AFP) and the method is in accordance with EXAMPLE 3 of WO 96/37516, incorporated herein by reference.

### EXAMPLE 20 TO 26

The TAB is intact antibody ZCE-025 (see example 1) specific to CEA. It is directly Iodinated with 131 I, using the chloramine-T (CT) method following the procedure of J. A. Carrasquillo et. al., Cancer Treatment Reports, Vol. 68, No 1, pp. 317-328, January 1984.

The TAB-131 I is administered in a dose containing 5 to 400 mCi radioactivity. The corresponding amount of Iodinated Mab is 0.65-52 mg. The other parameters of Tab administration are identical to those in EXAMPLES 1-6 In the above examples the 131 I TAB is used for TREATMENT. The dose used for diagnostics is 5 to 15 mCi. The procedures for adsorption of CA, and/or CA-NAB and/or NAB and/or TAAA and/or TGFβ and/or p 15E and/or other tumor suppression including tumor immunity suppression factors are identical to those described in EXAMPLES 1-6.

The adsorbent of TAB-131 I in the ECA column is an antiidiotypic antibody to the TAB, or an antiisotypic antibody (for example sheep anti mouse IgG, when the TAB is a Mouse IgG antibody)

In all the EXAMPLES 1-26, wherein prior to Step 1 ECA, the treated organism is administered a species (TAB, antibody to TAA, antibody to NAB antibody to a tumor suppression factor, such as TGFβ3 and p 15 E, for example or any other TSF including TISF) aimed at production of a complex between the administered species and a targeting-inhibitor species or a tumor immunosuppressor species. Rather then administering the species to the treated subject, the species (such as the TAB, antibody to TAA etc. supra), which antibodies comprise an Fc piece (preferably the antibodies are intact monoclonal antibodies) The species is bound to the Protein A adsorbent in the ECA column, rather then being administered to the organism, following the general method of using ECA with Protein A or Protein G bound to a specific antibody, in accordance with U.S. Pat. No. 5,753,227 incorporated by reference.

### EXAMPLES 27 TO 52

The procedure for the adsorption of CA and/or CA-NAB and/or NAB and/or TAAA is identical to examples 1-26. The TAB is identical to the TAB of EXAMPLES 20-26, except that the TAB is treated in accordance to the procedures disclosed in U.S. Pat. No. 6,251,394, for the labeling of the TAB for post TAB administration adsorption. This patent is incorporated herein in its entirety, by reference. The labeling of TAB is preferably with Biotin.

The TAB may contain any of the therapeutic or diagnostic ligands described in the above patent (as well as those described in the current disclosure in its entirety, including, but not limited to EXAMPLES 1-26 above and those disclosed in PCT WO 96/37516).

The subject being treated for therapeutic or diagnostic purposes is a human subject with a malignant melanoma tumor which is positive for the p97 surface glycoprotein antigen. The TAB is the monoclonal antibody 96.5 specific to p97. The TAB is labeled with 131 I and conjugated to Biotin in accordance with U.S. Pat. No. 6,251,394. See column 8, line 66 to column 9, line 19:
“The monoclonal antibody was the 96.5 (mouse IgG2a) specific for p97, a cell surface glycoprotein with the molecular weight of 97,000 present on 60-80% of human melanoma. The tumor model has been described in detail (Ingvar. C. et al., Nucl. Med 30. 1989, 1224).

2. Conjugation and Labeling of Monoclonal Antibodies.
The monoclonal antibody 96.5 (330 μg) was labeled with s 37 MBq iodine-125 (125%, using the Chloramine-T method. By elusion on a Sephadex G25 column (Pharmacia PD 10) the fraction containing the labelled protein was collected and used for the conjugation. The labelling efficiency of the ¹²⁵I, 96.5 was around 70%. The radiolabelled monoclonal antibody was conjugated with biotin by mixing 500 μg of antibody with 41 μg of N-Hydroxysuccinimido-biotin (NHS-biotin) in 0.1 M NaHCO3, 0.15M NaCl with 10% DMSO. The mixture was incubated for 1 h at room temperature, followed by overnight incubation at 4° C. The ¹²⁵-McAb-biotin conjugate was separated from free biotin-reagent by gelfiltration on a Sephadex G25 column, equilibrated with PBS (10 mM Sodium phosphate, 0.15 M NaCl, pH 7.3). The conjugate was stored at 4° C. until used.”

The dose administered to the subject is the same as in examples 20-26 (5-400 mCi, 0.65-52 mg TAB). 4 to 48 hours, preferably 12 to 24 hours after injection of the TAB, the subject is treated by passing his blood through an Avidin adsorption column, preferably Mitradep® column produced by Mitra Products, Inc.

The length of adsorption is in most cases between 1 hour and 10 hours, most preferably, between 2 hours and 4 hours and depends on the individual case, including body weight of the subject and dose of TAB, and can be determined by a person skilled in the art without undue experimentation. The volume of plasma treated is between 1 and 6 plasma volumes, most preferably between 2 and 4 plasma volumes. The flow rate is between 10-50 ml/min. A scintillation camera is used for imaging, when imaging is desired.

In the step of removal of CA, CA-NAB, ATAA, NAB, TGFβ, P 15E, Sialomucin and other molecular and cellular tumor suppression factors (TSF) (included in TSF are tumor immunity suppressor factors (TISF), such as: sR TNF alpha and beta, sR IL 1, 2, and 6, and sR INF gamma; whenever TSF is mentioned, TISF are also included unless specifically excluded), rather than using Protein A as adsorbent, Protein A bound to a specific antibody to CA, CA-NAB, ATAA, NAB, and TSF (TSF molecules and/or cells TH2 suppressor cells epitopes, for example), may be used, wherein the adsorbent is Protein A bound to a specific antibody, as disclosed in U.S. Pat. No. 5,753,227, incorporated by reference.

Protein A bound to specific antibody, can be used instead of, or in addition to free Protein A adsorbent (e.g. Protein A unbound to specific antibodies).

Alternatively to the above option, in the step of removal of CA, CA-NAB, ATAA, NAB and other molecular and cellular TSF including TISF, a FIRST affinity adsorbent, other than Protein A or Protein G, can be used for binding to the FIRST affinity adsorbent a SECOND affinity adsorbent, specific to the TSF.

FIG. 1 shows an ECA device embodying this part of the invention. The device 1 is illustratively attached in the blood circulatory system of a mammal 3, by catheters 5 and 7. The device 1 includes a column 8 filled with a matrix in the form of beads 9. As shown in FIG. 2, a FIRST adsorbing ligand 11 having a binding compound portion 13 having affinity for a binding partner, is covalently bound to the matrix 9 in the column 7, and a SECOND affinity adsorbent 15, including a portion 17 specific to the chemical or molecular species to be removed by adsorption is bound to the FIRST adsorbent by a portion 19 of the affinity adsorbent constituting the binding partner. The FIRST adsorbent 11 can be Protein A, for example and the SECOND adsorbent 15 can then be an antibody specific to the species to be removed. Alternatively, and preferably, the FIRST adsorbent 11 in the ECA column is, or comprises as its binding compound portion 13, Avidin or strepavidin to which is bound a SECOND specific adsorbent 15, consisting of biotinylated antibody to the species to be removed (STBR) (such as, for example, CA, TAB, ATAA, CA-NAB, NAB, TSF (as previously indicated TSF includes TISF); in this preferred embodiment, the antibody portion of the SECOND adsorbent constitutes the portion 17, and the biotin portion constitutes the binding partner 19. When the TISF is a soluble receptor (such as, for example, sR TNF alpha, sR TNF beta, sR IL (1, 2, or 6) and sR INF gamma) the adsorbent for the soluble receptor TISF can be either a biotinylated antibody to the soluble receptor or it can be the biotinylated respective cytokine (TNF alpha, TNF beta, IL 1, 2, and 6 and INF gamma). The biotinylated cytokine may be directly bound to the Avidin that is covalently bound to the matrix in the column, or alternatively the cytokine may be first covalently bound to a biotinylated carrier molecule such as a protein, a peptide, a polysaccharide molecule or a polyamine or another polymer molecule that is bound to the Avidin in the column. Multiple different SECOND adsorbents, each specific to at least one STBR can be bound to the FIRST adsorbent. Alternatively, some of the STBRs, for example, CA, NAB, CA-NAB, ATAA, TAB can be removed by using only one adsorbent such as Protein A or Protein G, or a specific antibody to the STBR covalently bound to the matrix in the column. Other STBRs can be removed by the use of a FIRST and a SECOND adsorbent as described supra; for example, a FIRST adsorbent is Protein A and a SECOND adsorbent is an antibody specific to the STBR, or a FIRST adsorbent is Avidin and a SECOND adsorbent is a biotinylated antibody specific to the STBR.

The use of Avidin-Biotin combination, wherein the FIRST adsorbent is Avidin, was proposed by J Tennval et al., Cancer Suppl., Vol. 80, number 12, pp. 2411-2418, Dec. 15, 1997, for the removal of ATAA, by adsorbing to the Avidin in the ECA column biotinylated TAB. In accordance with the current invention, this approach can be utilized to remove any molecular or cellular TSF, by binding to the Avidin in the ECA column one or more biotinylated antibodies specific to one or more molecular or cellular TSF. It should be realized that the method of utilizing a SECOND specific adsorbent, bound non-covalently (e.g. bound by ligand) to a FIRST adsorbent that is covalently bound to a matrix, is not limited to Protein A-specific antibody or to Avidin-Biotinylated specific antibody pairs. Various pairs of affinity ligands can be used as the FIRST adsorbent and the SECOND adsorbent. The FIRST adsorbent, for example, could be an antigen or a hapten covalently bound to a matrix, or dinitrophenyl (DNP) bound to a matrix, preferably through a spacer, or a carrier molecule, such as albumin (e.g., albumin-DNP conjugate bound to the matrix). The SECOND adsorbent can then be a hybrid antibody specific to both DNP and the TSF. The FIRST adsorbent can be an antibody to DNP and the SECOND adsorbent an antibody to TSF covalently bound to DNP. The FIRST adsorbent can be an enzyme bound covalently to the matrix in the ECA and the SECOND specific adsorbent will then be an enzyme substrate or enzyme inhibitor conjugated to an antibody specific to TSF (or to another molecular or cellular species targeted for removal). Similarly, any of other known affinity pairs such as, for example, those listed in U.S. Pat. No. 6,251,394, column 7, lines 54 to 67, can be used, including Lectins/saccharide residues (e.g. lectin from Sambueus nigra/beta-D-gal(1-4)-D-glc), Enzyme/enzyme inhibitors (e.g. D-Alanin carboxypeptidase from B. subtilis or E. coli/6-aminopenicillanic acid or p-aminobenzylpenicillin, or e.g. Dehydrofolate reductase/aminopterin or amethopterin), or protein/co-factors. (e.g. Intrinsic factor/vitamin B12 or cobalamin). It will be understood that with any affinity binding pair, either one of the affinity binding pair may be the FIRST adsorbent and the other of the pair may be the SECOND adsorbent.

Such specific adsorption methods using a FIRST and a SECOND adsorbent can be used not only in ECA treatment, but also in adsorption-based purification or diagnostic methods, to remove any molecular or cellular species from a fluid, including but not limited to biological fluids. As previously noted: whenever “TSF” is mentioned it includes “TISF”.

### EXAMPLES 53-56

These EXAMPLES are identical to EXAMPLES 4-8 except that the TL is the anti-cancer drug calicheamicin. The subject treated is a human being having an acute myeloid leukemia positive for the CD33 antigen. The TAB is recombinant engineered human anti-CD33. The TAB is conjugated to the calicheamicin in accordance to L M. Hinman et al., Cancer Research, Vol 53, pp. 336-3342, Jul. 15, 1993. 3336-3342. The dose of administered TAB-Calicheamicin conjugate is 6-9 mg protein/m2 (E L Sievers et al., Blood Vol. 93 (11), Jun. 1, 1999).

### EXAMPLES 57-65

These EXAMPLES are identical to EXAMPLES 1-8, except that the Targeting molecule, in these examples is a non-immunologic OTP, the hormone peptide somatostatin. The treated subject is a human being having a cancer with high concentration of somatostatin receptors, as determined by biopsy. (C. Casini Raggi et al., Clin. Cancer Res., Vol. 8 (2), pp. 419-427, Feb. 8, 2002.) Adriamycin is conjugated to the somatostatin in accordance with A. Nagy et al., Proc. Natl. Acad. Sci. USA, Vol. 95, pp. 1794-1799, 1998. The dose of the conjugate is calculated to contain 30 mg to 75 mg Adriamycin/m² body surface. The post administration of Somatostatin-Adriamycin conjugate clearance from BC and HC is done with an ECA column containing antibody to somatostatin bound to Protein A. Alternatively, the Somatostatin is conjugated also to Biotin in accordance with C M Eppler et al., J. Biol. Chem., Vol. 267 (22), pp. 15603-12, Aug. 5, 1992 and the conjugate administered to the subject is Biotin-Somatostatin-Adriamycin and the ECA is done with an Avidin column, for example, Mitra® ECA column. The subject is treated after post-conjugate administration so as to treat 1-5 plasma volumes. ECA is started 1-48 hours after Biotin-Somatostatin-Adriamycin is administered, preferably 2-24 hours. The length of post conjugate administration is 1-4 hours. The flow through the column is 20-50 ml/min.

### EXAMPLES 66-70

These EXAMPLES are identical to EXAMPLES 63-67, except that the subject being treated is administered a Yttrium-90 labeled somatostatin analog prepared according to A Otte et al., The Lancet, Vol 351, pp., 417-18, Feb. 7, 1998. The dose of the conjugate is 25 mCi to 200 mCi.

### EXAMPLE 71

A conjugate of Avidin with Mab ZCE-025, which is specific to CEA antigen is prepared according to H. P. Kalofonos et. al., The Journal of Nuclear Medicine, Vol. 31, No 11, pp. 1791-1796, 1990, except that the Mab ZCE-025 is substituted for Mab HMFG1. The subject being treated is having a tumor positive to the CEA antigen and has circulating CEA antigen in his blood. As determined by standard methods known in the art and available commercially. In Step 1: The subject is administered intravenously (1-4 min) 0.1 mg protein/Kg to 2 mg protein/Kg of the Mab-Avidin conjugate. In Step 2: The subject is treated with ECA with a Biotin Adsorbent Column (BAC). The BAC ECA starts 0.5 h to 48 hours, preferably 2-24 hours after the administration of the Mab-Avidin conjugate. 1-4 volumes of plasma are treated. The length of the ECA is 1-4 hours. The flow rate is 20 ml/min to 50 ml/min. For its use as the adsorbent in the ECA Biotin is covalently bound to a macromolecular carrier, such as human serum albumin (HSA), using the method described in U.S. Pat. No. 6,251,394, except that the albumin is substituted for the antibody. Albumin labeling with Biotin can also be done, following the method described in Harlow and Lane supra, pp. 340-341, substituting the HSA for antibody. The Albumin-Biotin is covalently bound to cyanogen bromide Sepharose 4B beads available from Pharmacia. As an alternative to binding of Albumin-Biotin to cyanogen bromide activated Sepharose, the binding of the biotinylated albumin to Sepharose can be done by using Avidin-Biotin binding. The albumin is biotinylated, so as to have sufficient Biotin moieties conjugated to the albumin, thus leaving sufficient number of Biotin molecules in the ECA column to be available to bind to Avidin in the Mab-Avidin conjugate. In Step 3, the subject is given intravenously 0.5-10 mg protein of the conjugate Biotin-Human Serum Albumin (HSA)-¹³¹I in 1-5 ml volume of 8.4% Sodium bicarbonate. Human serum albumin is directly Iodinated with ¹³¹I according to E. Harlow and D. Lane: Antibodies A Laboratory Manual, Cold Spring harbor Laboratory Pub., pp. 324-329, except that Human Serum Albumin is substituted for the antibody for the direct Iodination. The preferred Iodination is by the Chloramine T method (E Harlow and D Lane supra, 328-329).

In Step 4: 0.5-48 hours, preferably 2 hours to 24 hours following the administration of the BiotinHSA-131 I-conjugate, the subject is treated by ECA column, that includes Avidin as the adsorbent, Preferably the Avidin CEA column is the Mitra®) ECA column. Length of ECA is 1-4 hours, Flow rate in the ECA is 20-50 ml/min.

The purpose of Steps 1 and 2 is to remove CA, CA2-NAB and TAA from the BC and HC and from the Interstitial Fluid of TC, by specifically labeling CA CA2-NAB and TAA, with the TAB-Avidin conjugate and adsorbing the complexes TAB-Avidin-CA TAB-Avidin-CA2-NAB and TAB-AvidinTAA by adsorption to the Biotin adsorbent in the Biotin ECA column. The Mab in the TAB-Avidin conjugate can be an intact antibody, antibody fragment, including synthetic fragment, and fragment produced by genetic engineering techniques. In addition to the removal of CA, CA2NAB and TAA, removal of any other molecular and/or cellular species can be accomplished by the labeling of the species with a conjugate of Avidin that is conjugated to an antibody to the species, such suppressor species include: NAB, CA-NAB2, Transforming Growth Factor beta (TGFβ) p15 E factor, Interleukin 10 (IL-10), Prostglandin E2 (PGE2), Mucin, Suppressive E Receptor (SER), Immunosuppressive acidic protein (IAP) and adhesion molecules. (K. E. Hellstrom and I. Hellstrom, Encyclopedia of immunology, supra, and C Botti et al., Int. J. Biol. Markers, Vol. 13 (2), pp. 51-69, 1998.) sR TNF alpha, sR TNF beta, sR IL-1, sR IL-2, sR IL-6, sR INF gamma (M. R. Lentz; U.S. Pat. No. 6,231,536).

A labeled affinity targeting molecule can be administered to the subject being treated to affinity label any molecular or cellular species, in particular in the BC but also in the HC and the Interstitial fluid component of the TC, provided that the species targeted for affinity labeling is in equilibrium between the BC, HC and TC compartment (unless the removal is desired only from the BC or from the other treated biological fluid compartment, such as peritoneal fluid CSF or lymphatic fluid, when this fluid is treated in the ECA device, when equilibrium with HC and TC is not required. It should be realized that usually, these species will be in a concentration equilibrium between the various compartments). With respect to the species that it is desired to remove in the treatment (or diagnosis) of cancer, in addition to what was listed above: cellular species include TH2 suppressor T cells that suppress the immune destruction of tumor cells. It will be realized that the disclosed “labeling-based adsorption”, will have applications other than in the treatment of cancer, to remove any endogenous or exogenously administered or invading cellular or molecular species, such as autoantibodies in the treatment of autoimmune disease, sepsis associated factors, such as tumor necrosis factor, leukotrienes, bradykinin and Interleukin 2, in the treatment of sepsis; viruses and bacteria as well as protozoa in the treatment of infectious diseases; toxins: e.g. tetanus toxin, botulinum toxin, or ricin toxin (in particular ricin A peptide) for example (J. L. Middlebrook and R. B. Dorland, Microbiological Rev., September 1984, Vol. 48(3), pp. 199-221, or any other exotoxin or endotoxin such as E Coli endotoxin. Endotoxins are typically present in the outer membrane, and are released upon death and lysis of the bacteria (C. Galanos, in Encyclopedia of Immunology, I. M. Roitt and P. J. Delves eds., Academic Press, 1992, pp. 500-01, and A. N. Neely. et al, J. Burn Care Rehabil, 2002, September-October, Vol. 23, No 5, pp 333-40); an antibody to exotoxin or endotoxin, or any other MCS can be used for affinity labeling or as the adsorbent in the ECA column. can be used as the adsorbent.

Any chemical or cellular species to which a known affinity ligand is available or can be produced, including chemical species taken by accidental or intentional overdose, chemical agents utilized in bioterrorism and biological and chemical weapons, such as blister agents (mustard), sarin and other “nerve agents,” and any agent to which an antibody, antibody fragment, or synthetic analog of an antibody binding site are known or can be produced, as well as any agent for which an affinity adsorbent ligand that is not an immunological adsorbent (is not an antibody or antibody-based) is available or can be produced may also be removed. Specific antibodies for chemical agents that have been produced include, for example, antibodies to ricin toxin (H. F. Shyu et al., Hybrid Hybridomics, 2002, February, Vol. 21(1), pp. 69-73) and antibodies to sulfur mustard (C. N. Lieske et al, Immunol. Lett. 1992, February, Vol. 31(2), pp. 117-22).

The affinity adsorbent for the TSF (including TISF, including molecular and cellular TISF) as well as the affinity adsorbent for MCS, such as, for example, an antibody to Botulinum Exotoxin or an antibody to Tetanus Exotoxin (including antibody fragment, including synthetic fragments and synthetic or genetically engineered antibody binding site analogs.) Other affinity adsorbents can also be used, such as, for example, binding sites of Botulinum Toxin (N. M. Bakry et al: Infec. Immunol., Vol 65 (6) pp 225-32, 1997), including synthetic binding sites and synthetic analogs of binding sites. The affinity adsorbents can be incorporated in the ECA device, using any of the methods disclosed in U.S. Pat. Nos. 6,039,946 and 4,813,924, such as being covalently bound to a matrix (such as a natural polymer or synthetic polymer, as well as silica. Alternatively, the affinity adsorbent can be encapsulated in a microcapsule or macrocapsule, or can be incorporated or attached to a macrosphere or microsphere. When the affinity adsorbent is an antibody or an Fc containing antibody fragment, it can be bound to Protein A or Protein G through Fc non-covalent (by ligand) binding. When the adsorbent is a biotinylated, or Avidin-bound affinity adsorbent, it can be bound non-covalently (by ligand) to Avidin or Biotin, respectively, which are covalently bound to a matrix in the column. Other utilizations include affinity labeling for ECA of specific cellular and/or molecular species that are collected or harvested for therapeutic use, in the same subject, from which they are removed, or in another subject: For example, removal of cytotoxic T cells and natural killer cells (lymphokine activated killer cells (LAK) and tumor infiltrating Lymphocytes (TIL)), for in vitro activation and expansion, followed by re-administration to the subject, in the treatment of cancer, e.g. in vitro stimulation of harvested T and NK cells by treatment with lymphokines, in LAK or TIL cell treatment of cancer (See for example J. J. Sussman et al., Ann Surg Oncol, Vol 1 (4), pp. 296-306, 1994 and S A Rosenberg et al., Science, Vol. 233(4770), pp. 1318-21, September 1986). The only requirement for such affinity adsorption harvesting is the availability or the ability to produce specific affinity labeling reagent that is specific to the target molecular or cellular species, and the ability to conjugate the targeting species to an affinity marker, such as Avidin (or Biotin), for example. Markers of LAK and TIL cells include, for example, CD3+, CD56+, DM1, VGO1 and LAK1. (R E Herberman; Encyclopedia of Immunology, I M Roitt and P J D elves, Eds. Academic Press 1992, pp 1013-15.) It would be obvious that the method can be modified by, for example, the use of Biotin for affinity labeling and use of Avidin as adsorbent in the ECA, or the use of affinity pairs other than Avidin Biotin, for example: anti-hapten antibody—hapten, enzyme-substrate and the like. For example, in LAK and/or TIL based treatment, an antibody, such as biotinylated antibody specific to LAK and T cell markers, are used as the adsorbents or a tumor specific antigen (or a synthetic analog of the tumor specific antigen) to which the TIL has affinity, can be used as the adsorbent. One significant advantage of the proposed affinity labeling, is that it can be possible to use one single ECA device (Avidin-ECA, Biotin-ECA, for example) to remove many different species from the subject, by using different specific affinity labels targeted to the species to be removed and adsorbing them on the single device used for the ECA step of the method. Alternatively, the different species can be removed at different times or at the same time. The affinity label specific to the species to be removed can either be administered to the subject being treated prior to the ECA step, or preferably the affinity labels are not administered to the subject being treated but are incorporated in the ECA device itself. For example, when the affinity labels are biotinylated antibodies specific to the species to be removed, and the device is an Avidin-ECA device.

For example, if the species is TAA it can be targeted by TAB-Avidin or by Anti-idiotypic antibody to TAA and removed by a Biotin-ECA. If, the species is oxidized LDL it can be affinity labeled by administering a conjugate of Avidin-Antibody to oxidized LDL and removed by ECA with Biotin as the adsorbent. Pairs of affinity labels and their counterparts are known in the art and are disclosed, for example, in U.S. Pat. No. 6,251,394, column 6, line 7 to column 8, line 53. Many specific ligands in the Avidin-specific ligand conjugate are known and many are available from commercial sources. Generally, such specific ligands are known by, or can be produced by persons with ordinary skill in the art, following established methods and without undue experimentation. For example, if the specific targeting ligand is a Mab, many of the molecular and cellular species, that are affinity targeted for removal have known Mabs that are specific to them and many of them are available commercially. For example, Mabs to CEA and other tumor antigens mentioned in the current application have been produced. Alternatively, such Mabs can be produced, without undue experimentation by those skilled in the art, by use of hybridoma Mab production techniques. As an alternative to administering affinity labels to the subject, the TSF, including TISF affinity labels as well as affinity labels specific to any endogenous or exogenous administered or invading cellular or molecular species, as detailed above (all such molecular or cellular species will be referred to as MCS) can be incorporated in the ECA column, utilizing an ECA method that incorporates as adsorbents a FIRST and a SECOND adsorbent. For example the FIRST adsorbent may be Avidin or Protein A covalently bound to the matrix in the ECA column and the “SECOND” adsorbent may be a plurality of biotinylated antibodies (when Avidin is the FIRST adsorbent), or untreated antibodies (when Protein A is the FIRST adsorbent) that are specific to at least two TSF species, with, or without use of regeneration of the adsorbents. Use of double stage labeling of a tumor for radiolabeling a cancer was reported in H. P. Kalofonos et al., The Journal of Nuclear Medicine, Vol. 31, no 11, pp. 1791-1796, November 1990. The authors labeled a lung cancer with a conjugate of a Mab specific to the cancer antigen, that was conjugated to Strepavidin and administered Biotin-¹¹¹In to radio-label the tumor for diagnostic imaging. The authors did not disclose or hint to the possible use of such labeling for the ECA of molecular and cellular species. Matrixes other than Sepharose can be used to produce matrix-biotin conjugate for use in ECA. For example, G. Paganelli et. al. disclose the production of biotinylated Nitrocellulose and biotinylated Polystyrene (G. Paganelli et al., Int J. Cancer Suppl. 2, pp. 121-125, 1988.).

Avidin ECA column can be used with whole blood rather then plasma, thus simplifying the adsorption as disclosed by J. Tennvall et al., Cancer Vol. 80, No 12 (Suppl.) pp. 2411-2418, Dec. 15, 1997.

Following ECA on the Biotin adsorbent column, the subject is administered a VL or TL conjugated to Biotin (it should be realized that the use of Avidin and Biotin in the example can be reversed; with the sequence of Step 1: Administration of Biotin-TAB, Step 2: ECA on Avidin column Step 3: Administration of TL-Avidin or VL-Avidin conjugate. Step 4 ECA on a Biotin column. It would also be realized that affinity pairs other than Avidin biotin can be used instead of Avidin-Biotin pair (such as, for example: Hapten-Anti hapten antibody, Enzyme-substrate, Enzyme-Enzyme inhibitor.) The VL-Biotin conjugate can be Biotin-¹¹¹In and is prepared and administered to the subject according to Kalofonos et al., supra: Biotin covalently conjugated to diethylenetriaminepentaacetic acid (DPTA) is obtained from Sigma Chemical Co., St. Louis, Mo., and chelated to ¹¹¹In as described in Kalafonos et al. supra.

### EXAMPLE 72

The subject is a human with CEA positive cancer, as described in Example 71. In Step 1 the subject is administered a conjugate of Avidin with Mab ZCE-025 specific to CEA.

In Step 2, the subject is treated with ECA, incorporating biotin as the adsorbent. The purpose of this step is to remove CA, C2-NAB and TAA.

In Step 3, An anti-cancer drug, including, but not limited to one or more of the anticancer drugs disclosed in PCT WO 96/37516 as well as the anticancer drugs disclosed in U.S. Pat. No. 5,527,528, incorporated herein by reference is incorporated in a liposome prepared as described in U.S. Pat. No. 5,527,528, in particular, example 3, for the preparation of Avidin coated liposome omitting the last step of incubating the Avidin coated liposomes with biotinylated antibodies. Optionally the Avidin is bound to the Biotin on the liposome wall through a spacer. Suitable spacers are disclosed, for example, in K Hashimoto et al., Biochim Biophys Acta, Vol. 856 (3), pp. 556-65, Apr. 25, 1986. The anticancer drug incorporated into the liposome in the Example is adriamycin.

In Step 4, the subject is treated by ECA with Biotin incorporated as the adsorbent, to remove liposomes from the BC that did not reach or become attached to the cancer.

Optionally, Step 2, and/or Step 3 can be omitted.

Alternatively the liposomes are biotinylated liposomes of U.S. Pat. No. 5,527,528, optionally with the Biotin connected to the liposome wall with a spacer arm, the adsorbent in the ECA of Step 2 is Avidin and the adsorbent in the ECA in Step 4 is Biotin.

### EXAMPLE 73

The subject is the same as in example 72. In Step 1, he is administered Mab ZCE-025, specific to CEA conjugated to Biotin following the method of U.S. Pat. No. 5,527,528. In Step 2, the subject is treated with ECA incorporating Avidin as the adsorbent. In Step 3, the subject is administered Ricin A conjugated to Avidin (L. K. Mahal et al., Science, Vol. 276, pp. 1125-1128, 16 May, 1997.)

In Step 4, the subject is treated with ECA, incorporating Biotin as the adsorbent.

In all of the Examples wherein the administered species is Avidin conjugate of TAB, or the administered species is Avidin conjugate of a non-immunologic targeting molecule, in order to remove from the treated subject, when desired any of the species that would inhibit targeting and/or species that would in general suppress the immune destruction, or non-immune mechanism destruction of the tumor, such targeting inhibitors (TIF) and/or tumor destruction inhibitors (TIF, including TISF) can be removed from the BC, HC and TC by incorporating in the ECA adsorption column one or more adsorbents that have specific affinity to the targeting inhibitor or tumor destruction suppressive molecular and cellular species. This can be accomplished by incorporating such specific adsorbents in a single column, or in different columns, connected in parallel or in series, as disclosed in U.S. Pat. No. 5,753,227. When the ECA incorporates Avidin as the adsorbent, the specific adsorbent added to the ECA Avidin column is a Biotin conjugate of a specific affinity ligand (such as Biotin-Mab specific to CA, and/or ATAA and/or NAB and/or any of the other suppressors as disclosed in Hellstrom and Hellstrom, supra, and in Botti et al., supra). When the adsorbent in the ECA column is Biotin, the Specific adsorbent added is Avidin-Mab specific to the CA and/or AT AA and/or NAB, or other target inhibitors or tumor destruction inhibitor (TSF including TISF) cellular or molecular species, as above.

### EXAMPLE 74

In any of the Examples 1-73, in the step of removal of specific species from the blood circulatory system or from other biological fluid, such as, peritoneal and CSF, the specific Protein A-specific intact antibody adsorbent ligand, can be used for the removal of any substance from a biological fluid source, and for the application of the current invention, can be used for the adsorption-removal of any chemical species that may interfere with targeting, such as CA, CA-NAB, NAB, ATAA, or any molecular or cellular species that suppresses the Immune Destruction, or Non-Immune destruction of the tumor (TSF, including TISF). The specific adsorbent is based on the method disclosed in U.S. Pat. No. 5,753,227. Said patent is incorporated herein in its entirety. In accordance with the current example, intact antibodies, or antibody fragments, containing Fc, wherein said antibodies or Fc containing fragments are specific to an epitope on the molecular or cellular species that is to be removed from the biological fluid and BC, HC and TC are added to a Protein A extracorporeal column, such as Immunosorba® or Prosorba®, or any other Extracorporeal column that contains Protein A, bound to matrix or encapsulated, as disclosed for example in U.S. Pat. No. 5,753,227, supra. Such antibodies or Fc containing fragments, in the current example are selected from antibodies, or fragments specific to the molecular and cellular species, that inhibit targeting or suppress tumor destruction as disclosed in the current application and in Hellstrom and Hellstrom and Botti et al., and M R Lentz; Therapeutic Apheresis Vol. 3 (1) p 40-49, 1999.

As disclosed in U.S. Pat. No. 5,753,227 and also detailed in Harlow and Lane supra, pp. 519-523, the antibody or fragment will bind to the protein A in the ECA through the Fc of the antibody or fragment, thus producing a specific adsorbent ligand to specifically adsorb one or more of the targeting-inhibiting molecular species, or molecular or cellular species that inhibit or suppress tumor destruction. This can be accomplished by incorporating such specific adsorbents in a single ECA column, or in different columns, connected in parallel, or in series, as disclosed in U.S. Pat. No. 5,753,227.

### EXAMPLE 75

In any of the Examples 1-8 the tumor being treated with targeted TL, or visualized with targeted VL, is a Prostate Tumor, including but not limited to a prostate tumor with metastatic spread. The tumor antigens include for example Prostate Specific Antigen (PSA), Prostatic Acid Phosphatase (PAP), p 53 and HER-2neu (D. G. McNeel et al., J. Urol. 2000, November; Vol 164, No 5, pp 1825-1829) and prostate specific membrane antigen (PSMA) (M. J. Burger, Ont. J. Cancer, July 10, Vol. 100 (2): pp 228-37, and S. S. Chang and W. D. Heston, Urol. Oncol., 2002 January-February; Vol. 7(1) pp 7-12).

### EXAMPLE 76

Extracorporeal Removal of IL-6 from Plasma or Blood

In atherosclerosis (AS), IL-6 is elevated in the blood and systematically in the body, as well as AS plaques (ASP), particularly in unstable angina (UA). The adsorbent used in the ECA is a Mab specific to IL-6 (L. J. Cornfield and M. A. Sills, Eur. J. Pharmacol., Sep. 4, 1991, Vol. 202(1) pp. 113-5. The Mab is incorporated in ECA device. Suitable devices and matrixes, including suitable methods for covalent and non-covalent binding of the Mab to the matrix are those described in U.S. Pat. No. 4,813,924, example 13 and FIG. 7, and U.S. Pat. No. 6,039,946 when modified for use for only one Mab adsorbent. (It is noted that whenever Mab is mentioned, it includes fragments, including synthetic fragments and analogs.) The Mab can be encapsulated in a microcapsule or macrocapsule as described in U.S. Pat. No. 6,039,946; see in particular U.S. Pat. No. 6,039,946 col. 8 line 31 to col. 11, 1.60 and FIG. 4. When Mab to IL-6 is encapsulated according to the original method of L. Markus et al., Am. Heart J., Vol. 110(1), part 1, July 1985, pp. 30-39, either the modified macrosphere encapsulation as used in the patent, or the method as described in Markus et al. can be used for the encapsulation of the IL-6 Mab. Preferably the Mab is covalently bound to cyanogen bromide activated cross linked agarose as described in the patent except that Mab to IL-6 is substituted for the Mab specific to LDL.

Alternatively Mab to IL-6 can be bound in the ECA column by non-covalent binding to Protein A that is covalently bound to the matrix as disclosed in the patent. Alternatively the Avidin and Biotin columns can be used to provide matrix-bound Mab, specific to IL-6. The Mab to IL-6 is used instead of the Mab 96.5 specific to P97 antigen disclosed in the patent application and the biotinylated Mab to IL-6 is bound to Avidin in the Avidin ECA column, for example the Avidin column produced by Mitra Products (Mitradep® column) The biotinylated Mab is bound in the column by the high affinity ligand binding of the biotinylated Mab to the Avidin which is bound covalently to the matrix in the column. The plasma flow rate in the ECA device, is usually 20-35 ml/min (flow rate can vary, depending on the individual case and can be in the range of 5-50 ml/min. Depending on the individual case and the particular column used. Persons with skill in the art can determine without undue experimentation, the appropriate flow rate in the individual case.

The length of the each ECA treatment is typically between 1 hour and 17 hours and more typically between 2 hours and 4 hours, depending on the individual case, including, for example weight and age of the subject. The number and frequency of treatments will depend on the individual case and can be determined without undue experimentation, by persons with skill in the art. It will be understood that while in the Example, in the treatment of AS, Plasma (using a Plasma Separator on-line) or blood are treated, when the devices and the method of the invention are used for the treatment of diseases other than AS (such as autoimmune disease, rheumatoid arthritis, cancer and other inflammatory diseases) any biological fluid can be treated, for example, peritoneal fluid and cerebrospinal fluid. Methods to access such biological fluids are known in the art. However, in the treatment of AS, plasma and blood will be in the biological fluids treated. Clearly, other affinity pairs can be used. For example, use of a Biotin ECA column and Avidin bound antibodies.

### EXAMPLE 77

Removal of multiple molecular inflammatory factors (MIFs) in the treatment of AS, in particular AIS, utilizes methods and devices disclosed in U.S. Pat. No. 6,039,946, particularly as described in col. 8 line 31 to col. 13 line 62 and in FIGS. 4, 5 and 6. The methods and devices are essentially similar to Example 1, except that multiple adsorbents are utilized, with each adsorbent being specific to or selective for each one of the MIFs. The adsorbents are incorporated in the ECA in accordance with U.S. Pat. No. 6,039,946. The preferred adsorbents are antibodies, preferably Mabs, including fragments. When an Avidin column is used, the biotinylated antibodies are bound to the Avidin as described in Example 1, with respect to Mab specific to IL-6: the adsorbents incorporated in the ECA can be adsorbents for any of the MIFs, including but not limited to all the MIFs described in the background section above and to the species etiological in AS that are described in U.S. Pat. No. 6,039,946. Mabs are known to many of the MIFs and in any event can be produced by using techniques known in the art, for the production of Mabs.

The molecular species to be removed are selected from the following list, that intends to give examples and is not intended to limit the scope of the invention: IL-6, TNF alpha, Heat Shock Protein (HSP), antibodies to oxidized LDL (OxLDL), antibodies to HSP, CRP, LDL, triglycerides, IL-2, metalloproteinases, other proteinases, fibrinogen, creatine kinase, IL-1-Beta, IL-1-Ra, PDGF, angiotensin II, MCSF, and pregnancy associated plasma protein A (PAPPA). The parameters of ECA procedure, such as flow rate and length of treatment session, will be in the range of the parameters described in Example 1.

Clearly, rather than antibodies, other affinity ligands can be used instead, such as receptors and their synthetic peptides which include the binding site for the molecule to be adsorbed, including synthetic analogs of receptors, other selective adsorbents, such as dextran sulfate or heparin, or polyacrylic acid, for example, to adsorb LDL, substrate of enzymes (proteinases) or enzyme inhibitors, chelators for removal of metal containing species such as metalloproteinases, as examples.

### EXAMPLE 78

The treatment may be utilized by itself or in addition to ECA treatment as described in Examples 1 and 2. It involves the administration of antibodies, preferably Mabs, preferably chimeric or humanized and including fragments, synthetic fragments and analogs which are specific to one or more of the MIFs, for example the Mab specific to IL-6.

Also, in addition to ECA conventional anti inflammatory drugs, receptor antagonists and anti coagulants, for example and any other drugs used in the treatment of AS, in particular AIS, can be used in conjunction with the use of the ECA methods in all of the Examples given herein. In addition to conventional drugs, anti inflammatory cytokines, such as IL-10, TGF-Beta (K Mizzia et al., supra, M. Londei, in Encyclopedia of Immunology, I. M. Roitt and P. J. Delves Eds., Academic Press 1992, pp. 443-445), can be administered in conjunction with the treatment described in Examples 1-5, or as a stand-alone treatment.

### EXAMPLE 79

This treatment is primarily for non-acute stages of AS: The treatment utilizes Active Immunization with one or more of the MIFs administered in an immunogenic preparation.

The immunogenic MIF, is preferably administered in conjunction with an adjuvant, preferably an adjuvant known in the art o be suitable for administering to humans, for example, one of the adjuvants disclosed in U.S. Pat. No. 6,054,127. The immunogen may be incorporated in a liposome in order to increase its immunogenicity, as known in the art (G. Gregoridas, Trends in Biotechnology, Vol. 13, pp. 527-37, 1995). MIFs of MW below 10000 kD are preferably conjugated to a suitable carrier, as known in the art, for example KLH, Albumin or peptides.

### EXAMPLE 80

This method is used by itself, or in conjunction with any of the methods and devices described in Examples 1-4. The method involves the removal by affinity adsorption, of Cellular Inflammatory Factors (CIFs). Optionally this is done by Extracorporeal treatment of, blood, plasma, or in some situations, other biological fluids, but in most applications of the method is utilized by removing blood and treating it outside the body, without the use of ECA, and returning the treated blood to the treated subject. Any one, or more of the CIFs mentioned in the background section can be removed.

The preferred CIF to be removed, is CD4+ CD28null T lymphocytes, that are strongly implicated in the EP of AIS. In addition, or instead, other CIFs can be removed, including but not limited to the following: Th1 T cells, CD14 T lymphocytes, CD 40+ Platelets, CD8+ CD 116+ Cytotoxic T cells, CD11b+CD16+ Lymphocytes and CD56+ Lymphocytes. CIPs can be removed by affinity adsorption with specific antibodies, preferably Mabs and including fragments, as described in Provisional U.S. Patent application 60/374,715.

CIFs can also be removed by affinity adsorption with Mabs, utilizing the method described in B. L. Levine et al., J Hematotherapy, 1998, October, Vol. 7 (5), pp. 437, utilizing Mabs specific to one or more CIFs to specifically remove them. The system described in the paper above utilizes magnetic separation with antibody coated magnetic beads and a MaxSep® magnetic separator. Preferably, in the current Example, CIFs are removed using the Isolex 300i® system (Nexell Therapeutics), with the use of Mabs specific to the CIFs that are targeted for removal. The Isolex 300i® uses paramagnetic beads to which the specific Mab is bound, to remove the specific cells (CIFs), When it is desirable not to discard the CIFs, such as when it is desirable to collect them, the Isolex 300i® system provides a unique peptide release system to separate the cells from the Mab bound magnetic beads. Depending on the CIF to be removed either the removal system of the Isolex 300i® or another suitable peptide can be used, or the cells can be separated by other means, such as change in pH, mechanical shaking, change in ionic strength, or use of soluble cell receptor (epitope) including synthetic fragments of the receptor, including analogs. When in the Example the CIF targeted for removal is CD4+ CD28null T cells, it is desirable not to reduce unnecessarily general immune function of the organism being treated. In order to accomplish this aim, first CD4+ cells (both CD4+ CD28null and CD4+ CD28+) are removed by the Isolex 300 I® system, utilizing Mab specific to the CD4+ epitope. CD4+ cells are then eluted, as described above, The removed cells are then treated with beads that are bound to Mabs specific to the CD28 epitope, the bound cells are eluted: these are CD4+ CD28+ T cells. These cells are returned to the subject being treated. The CD4+ CD28null cells do not bind to the Mab-bound paramagnetic beads and are discarded, or collected for testing, if desired. Mabs specific to epitopes on CIFs can be produced by known hybridoma and other Mab production techniques, routinely used in the art. Many of the CIFs epitopes have known Mabs. For example, Mabs are known for CD28 epitope (S. Fretier et al., J Leukoc. Biol., 2002, February, vol. 71(2), pp. 298-94, S. D. Singh and C. G. Booth, 3 Immunol. Methods 2002, Vol. 260 (1-2) pp. 149-5⁶). Anti CD4 Mab is also known. B. Sawltki et al., Euro. J. Immunol, March 2002, Vol. 32(3) pp. 800-9. CD4+ T cells can also be removed by an extracorporeal adsorption system described by H. Onodera et al., Ther. Apher., 1998, Feb. 2(1), pp. 37-42.

### EXAMPLE 81

Removal of at least one of the molecular species that are etiological in and/or are involved in the pathogenesis of Idiopathic Dilated Cardiac Myopathy (IDCM)

The molecular species include autoantibodies specific to any of the following: the β1 Adrenergic Receptor, autoantibodies specific to oxidized LDL, ADP-ATP Carrier, alpha cardiac myosin heavy chain isoform, beta cardiac myosin heavy chain isoform, g protein coupled receptors, and heart mitochondria. The method utilizes methods and devices disclosed in U.S. Pat. No. 6,039,946, particularly as described in col. 8, line 31 to col. 13, line 62 and in FIGS. 4, 5 and 6, except that one or multiple adsorbents are utilized. When more than one adsorbent are utilized, each adsorbent being specific to or selective for each one of the molecular species. The adsorbents are incorporated in the ECA in accordance with U.S. Pat. No. 6,039,946. The preferred adsorbents are the respective biotinylated antigens, to which the Autoantibodies are specific or the adsorbents can be biotinylated antiidiotypic antibodies that are specific to the respective autoantibodies. The antiidiotypic antibodies are preferably Mabs, including fragments. When an Avidin column is used, the biotinylated antigens or the biotinylated antibodies are bound to the Avidin as described in Example 1, with respect to Mab specific to IL-6. When antiidiotypic Mabs are used as the adsorbents they can be produced by using techniques known in the art, for the production of Mabs. The parameters of ECA procedure: Flow rate, length of treatment session, will be in the range of the parameters described in Example One.

### EXAMPLE 82

In any of the examples 1-8, except that the tumor being treated with targeted TL, or visualized with targeted TL, is an Ovarian Tumor, including but not limited to an Ovarian Tumor with metastatic spread. The tumor antigens is CA-125 and the targeting antibody is Mab OC 125 (Goff, B A et al: Br. J. Cancer, Vol. 70, pp 474-480, 1994).

These examples are merely illustrative. Numerous variations, within the scope of the claims, will occur to those skilled in the art in light of the foregoing disclosure.

1. A method of removing at least one species from a mammal, the method comprising a first step of confining in a device a binding compound, the binding compound having affinity for a binding partner, a second step of preparing a plurality of affinity binders, each of said affinity binders comprising a first portion comprising the binding partner and a second portion adapted to bind selectively with a species, and thereafter a step of selecting at least one of the affinity binders and introducing said at least one affinity binder into the device so as to cause the binding partner to bind to the binding compound, and a step of connecting the device to a fluid source in a mammal.

2. (canceled)

3. The method of claim 1 wherein the binding device includes a semipermeable membrane for confining the binding compound.

4. The method of claim 1 wherein the binding compound is bound to a carrier.

5. The method of claim 4 wherein the carrier is selected from the group consisting of a wall of the device, a fixed matrix in the device, and a fill of beads or other granules.

6. The method of claim 1 wherein the second portions of the affinity binders are adapted to bind selectively with pathogenic species.

7. The method of claim 1 wherein the affinity binders comprise antibodies.

8. The method of claim 7 wherein the first portions of the affinity binders comprise Fc portions of the antibodies.

9. The method of claim 1 wherein the first portions of the affinity binders comprise avidin or biotin.

10. The method of claim 1 wherein the device is adapted for removing blood from a mammal, the step of connecting the device to a fluid source in a mammal comprising passing at least a part of the blood through the device, and returning at least a part of the blood to the mammal.

11. The method of claim 1 wherein the binding compound comprises Protein A or Protein G.

12. The method of claim 1, including a step of administering to the mammal a targeting species bound to a targeted species, wherein the targeted species is selected from a Treatment Ligand and a Visualization Ligand.

13. The method of claim 1 wherein the second portion of at least one affinity binder is adapted to bind selectively with a species comprising a targeting species bound to a targeted species.

14. The method of claim 12, including a step of extracorporeal adsorption of a species comprising a targeted species.

15. The method of claim 12 including a step of extracorporeal adsorption of a species comprising a targeted species bound to a targeting species.

16. The method of claim 12 wherein the targeted species is a treatment ligand.

17. The method of claim 16, wherein the targeted species is an anti-cancer drug.

18. The method of claim 17 wherein the anti-cancer drug is selected from a radioactive molecule, a radioactive ion, a radioactive atom, a biological toxin, adriamycin, and calicheamicin.

19. The method of claim 10, wherein the targeted species is a visualization ligand.

20. The method of claim 19, wherein the visualization ligand, comprises a radioactive molecule, a radioactive ion, or a radioactive atom.

21. The method of claim 20 wherein the targeting species comprises somatostatin or an analog of somatostatin.

22. A method of making a binding device comprising a first step of confining in the device a binding compound, the binding compound having affinity for a binding partner, a second step of preparing a plurality of affinity binders, each of said affinity binders comprising a first portion comprising the binding partner and a second portion adapted to bind selectively with a species, and thereafter a step of selecting at least one of the affinity binders and introducing said at least one affinity binder into the device so as to cause the binding partner to bind to the binding compound, wherein at least one of the affinity binders comprises a first portion having affinity to the binding compound and a second portion having affinity to at least one of the group consisting of TNF alpha, IL-6, CD3+ DR+ T cells, CD4+ CD28null T cells, CD3+, CD56+, DM1, VGO1, LAK1, CRP, INF gamma, CA, NAB, CA-NAB, TGF β, P15E, Sialomucin, TH2 T cell epitope, tumor infiltrating lymphocyte (TIL) marker, lymphokine activated killer cell (LAK) marker, Interleukin 10 (IL-10), prostaglandin E2 (PGE2), mucin, suppressive E receptor (SER), immunosuppressive acidic protein (IAP), adhesion molecules, sR TNF alpha, sR TNF beta, sR IL-1, sR IL-2, sR IL-6, sR INF gamma, heat shock protein (HSP), antibodies to oxidized LDL (Ab-OxLDL), antibodies to HSP, CRP, triglycerides, IL-2, metalloproteinases, other proteinases, fibrinogen, creatine kinase, IL-1-Beta, IL-1-Ra, PDGF, angiotensin II, MCSF, pregnancy associated plasma protein A (PAPPA), antibodies specific to any of the following: the β1 adrenergic receptor, ADP-ATP carrier, alpha cardiac myosin heavy chain isoform, beta cardiac myosin heavy chain isoform, G Protein coupled receptors, and heart mitochondria, oxidants, and toxins selected from the group consisting of botulinum toxin, tetanus toxin, ricin toxin, ricin A peptide toxin, endotoxin, sulfur mustard, prescription drugs, over-the-counter drugs, drugs of abuse, chemical poisons, and toxic metabolites thereof.

23. The method of claim 18, wherein the biological toxin is ricin A peptide.

24. The method of claim 12, wherein the targeted species is incorporated in a liposome.

25. The method of any of claim 12, wherein the targeting species has affinity for a tumor cell.

26. The method of claim 25, wherein the tumor is a cancer selected from ovarian cancer, prostate cancer, and colon cancer.

27. The method of claim 25 wherein the target binding site on the tumor cell is selected from: CEA, p97 epitope, CD33 antigen epitope, somatostatin receptor, p 53, Her-2neu, PSMA, and CA-125.

28. The method of claim 10, including a step of administering to the mammal a drug selected from a receptor antagonist and an anti-inflamatory cytokine.

29. The method of claim 10, wherein the device is an extracorporeal treatment device, the device being used for treatment of a mammal for a disease state selected from: cancer, atherosclerosis (AS), coronary artery disease (CAD), peripheral atherosclerotic vascular disease (PASVD), atherosclerotic cerebro vascular disease (ASCVD), acute ischemic syndromes, acute myocardial infarction (MI) and unstable angina (UA), inflammatory diseases, idiopathic dilated cardiac myopathy (IDCM), autoimmune disease, sepsis, infectious disease, diseases associated with exotoxins, diseases associated with endotoxins, autoimmune-associated vascular diseases, rheumatoid arthritis, drug overdose, drug intoxication, and poisoning with a chemical poison.

30. The method of claim 1, wherein the binding device comprises regeneration means, for regenerating the second portion of at least one affinity binder.

31. The method of claim 30, wherein the regeneration means comprise a solution.

32. The method of claim 31, wherein the solution comprises an acidic buffer.

33. In combination, a device having contained therein a binding compound bound to a carrier, the binding compound having affinity for a binding partner, and a plurality of affinity binders, each of said affinity binders comprising a first portion comprising the binding partner and a second portion adapted to bind selectively with a species, the second portions of each of said affinity binders differing from each other.

34. The combination of claim 33 wherein the device is an extracorporeal device including means for connecting the device to a fluid source in a mammal.

35. The combination of claim 33, wherein at least one of the affinity binders comprises a first portion having affinity to the binding compound and a second portion having affinity to at least one of the group consisting of TNF alpha, IL-6, CD3+ DR+ T cells, CD4+ CD28null T cells, CD3+, CD56+, DM1, VGO1, LAK1, CRP, INF gamma, CA, NAB, CA-NAB, TGF β, P15E, Sialomucin, TH2 T cell epitope, tumor infiltrating lymphocyte (TIL) marker, lymphokine activated killer cell (LAK) marker, Interleukin 10 (IL-10), prostaglandin E2 (PGE2), mucin, suppressive E receptor (SER), immunosuppressive acidic protein (IAP), adhesion molecules, sR TNF alpha, sR TNF beta, sR IL-1, sR IL-2, sR IL-6, sR INF gamma, heat shock protein (HSP), antibodies to oxidized LDL (Ab-OxLDL), antibodies to HSP, CRP, triglycerides, IL-2, metalloproteinases, other proteinases, fibrinogen, creatine kinase, IL-1-Beta, IL-1-Ra, PDGF, angiotensin II, MCSF, pregnancy associated plasma protein A (PAPPA), antibodies specific to any of the following: the β1 adrenergic receptor, ADP-ATP carrier, alpha cardiac myosin heavy chain isoform, beta cardiac myosin heavy chain isoform, G Protein coupled receptors, and heart mitochondria, oxidants, and toxins selected from the group consisting of botulinum toxin, tetanus toxin, ricin toxin, ricin A peptide toxin, endotoxin, sulfur mustard, prescription drugs, over-the-counter drugs, drugs of abuse, chemical poisons, and toxic metabolites thereof.

36. The combination of claim 33, wherein the device comprises regeneration means for regenerating the second portion of at least one affinity binder.

37. The combination of claim 36, wherein the regeneration means comprise a solution.

38. The combination of claim 37, wherein the solution is an acidic buffer.

39. The combination of claim 33 wherein at least one of the affinity binders comprises a second portion having affinity to a targeted species bound to a targeting species.

40. The combination of claim 39 wherein the targeted species comprises a radioactive molecule, a radioactive atom, or a radioactive ion.

41. A pharmaceutical preparation for the treatment of atherosclerosis comprising an immunogenic preparation of a molecular inflammatory factor (MIF) and a pharmaceutically acceptable pharmaceutical carrier.

42. A method of treating atherosclerosis, comprising administering to a mammal in need of such treatment the pharmaceutical preparation of claim 41.

43. An extracorporeal device, comprising at least one affinity adsorbent, said affinity adsorbent binding at least one exotoxin.

44. The device of claim 43 wherein the affinity adsorbent binds the exotoxin specifically.

45. The device of claim 43 wherein the affinity adsorbent is chemically bound to a matrix in said device.

46. The device of claim 43 wherein the device comprises at least two affinity adsorbents, said affinity adsorbents binding selectively to two different exotoxins, or an endotoxin and an exotoxin.

47. The device of claim 43, wherein the affinity adsorbent is selected from antibodies, antibody fragments, synthetic antibody binding site analogs, genetically engineered synthetic antibody binding site analogs, exotoxin receptor binding sites, synthetic or genetically engineered exotoxin receptor binding site analogs.

48. The device of claim 43 further comprising means for regenerating at least one affinity adsorbent.

49. The device of claim 48 wherein the regeneration means comprise a solution.

50. The device of claim 49, wherein the solution comprises a buffer.

51. The device of claim 50 wherein the buffer is an acidic buffer.

52. A method of removing a species from a mammal comprising introducing into the mammal an affinity binder which selectively binds the species, the affinity binder including a binding partner portion having affinity for a binding compound, and thereafter removing the affinity binder by capturing the affinity binder in a device having contained therein the binding compound, wherein the species is selected from the group consisting of TNF alpha, IL-6, CD3+ DR+ T cells, CD4+ CD28null T cells, CD3+, CD56+, DM1, VGO1, LAK1, CRP, INF gamma, CA, NAB, CA-NAB, TGF β, P15E, Sialomucin, TH2 T cell epitope, tumor infiltrating lymphocyte (TIL) marker, lymphokine activated killer cell (LAK) marker, Interleukin 10 (IL-10), prostaglandin E2 (PGE2), mucin, suppressive E receptor (SER), immunosuppressive acidic protein (IAP), adhesion molecules, sR TNF alpha, sR TNF beta, sR IL-1, sR IL-2, sR IL-6, sR INF gamma, heat shock protein (HSP), antibodies to oxidized LDL (Ab-OxLDL), antibodies to HSP, CRP, triglycerides, IL-2, metalloproteinases, other proteinases, fibrinogen, creatine kinase, IL-1-Beta, IL-1-Ra, PDGF, angiotensin II, MCSF, pregnancy associated plasma protein A (PAPPA), antibodies specific to any of the following: the β1 adrenergic receptor, ADP-ATP carrier, alpha cardiac myosin heavy chain isoform, beta cardiac myosin heavy chain isoform, G Protein coupled receptors, and heart mitochondria, a targeted species bound to a targeting species, and toxins selected from the group consisting of botulinum toxin, tetanus toxin, ricin toxin, ricin A peptide toxin, sulfur mustard, prescription drugs, over-the-counter drugs, drugs of abuse, chemical poisons, and toxic metabolites thereof.

53. A species-removing device having contained therein a binding compound attached to a matrix and an affinity binder bound by affinity binding to the binding compound, the affinity binder having affinity for a species selected from the group consisting of TNF alpha, IL-6, CD3+ DR+ T cells, CD4+ CD28null T cells, CD3+, CD56+, DM1, VGO1, LAK1, CRP, INF gamma, CA, NAB, CA-NAB, TGF 13, P15E, Sialomucin, TH2 T cell epitope, tumor infiltrating lymphocyte (TIL) marker, lymphokine activated killer cell (LAK) marker, Interleukin 10 (IL-10), prostaglandin E2 (PGE2), mucin, suppressive E receptor (SER), immunosuppressive acidic protein (IAP), adhesion molecules, sR TNF alpha, sR TNF beta, sR IL-1, sR IL-2, sR IL-6, sR INF gamma, heat shock protein (HSP), antibodies to oxidized LDL (Ab-OxLDL), antibodies to HSP, CRP, triglycerides, IL-2, metalloproteinases, other proteinases, fibrinogen, creatine kinase, IL-1-Beta, IL-1-Ra, PDGF, angiotensin II, MCSF, pregnancy associated plasma protein A (PAPPA), antibodies specific to any of the following: the β1 adrenergic receptor, ADP-ATP carrier, alpha cardiac myosin heavy chain isoform, beta cardiac myosin heavy chain isoform, G Protein coupled receptors, heart mitochondria, a targeted species bound to a targeting species, and toxins selected from the group consisting of botulinum toxin, tetanus toxin, ricin toxin, a ricin A peptide toxin, endotoxin, sulfur mustard, prescription drugs, over-the-counter drugs, drugs of abuse, chemical poisons, and toxic metabolites thereof.

54. The species-binding device of claim 53 wherein the affinity binder comprises an antibody, an antibody fragment, a synthetic antibody fragment, a synthetic antibody binding site, and a synthetic antibody binding site analog.

55. The species-binding device of claim 53 wherein one of the binding compound and the affinity binder comprises Avidin, and the other of the binding compound and the affinity binder comprises Biotin.

56. The species binding device of claim 54 wherein one of the binding compound and the affinity binder comprises Avidin, and the other of the binding compound and the affinity binder comprises Biotin.

57. The method of claim 1, wherein the binding device comprises regeneration means, for regenerating the second portion of at least one affinity binder.

58. The combination of claim 34, wherein at least one of the affinity binders comprises a first portion having affinity to the binding compound and a second portion having affinity to at least one of the group consisting of TNF alpha, IL-6, CD3+ DR+ T cells, CD4+ CD28null T cells, CD3+, CD56+, DM1, VGO1, LAK1, CRP, INF gamma, CA, NAB, CA-NAB, TGF β, P15E, Sialomucin, TH2 T cell epitope, tumor infiltrating lymphocyte (TIL) marker, lymphokine activated killer cell (LAK) marker, Interleukin 10 (IL-10), prostaglandin E2 (PGE2), mucin, suppressive E receptor (SER), immunosuppressive acidic protein (IAP), adhesion molecules, sR TNF alpha, sR TNF beta, sR IL-1, sR IL-2, sR IL-6, sR INF gamma, heat shock protein (HSP), antibodies to oxidized LDL (Ab-OxLDL), antibodies to HSP, CRP, triglycerides, IL-2, metalloproteinases, other proteinases, fibrinogen, creatine kinase, IL-1-Beta, IL-1-Ra, PDGF, angiotensin II, MCSF, pregnancy associated plasma protein A (PAPPA), antibodies specific to any of the following: the β1 adrenergic receptor, ADP-ATP carrier, alpha cardiac myosin heavy chain isoform, beta cardiac myosin heavy chain isoform, G Protein coupled receptors, and heart mitochondria, oxidants, and toxins selected from the group consisting of botulinum toxin, tetanus toxin, ricin toxin, ricin A peptide toxin, endotoxin, sulfur mustard, prescription drugs, over-the-counter drugs, drugs of abuse, chemical poisons, and toxic metabolites thereof.

59. The reagent of claim 41 wherein the Molecular Inflammatory Factor (MIF) is selected from the group consisting of TNF alpha, IL-6, CRP, INF gamma, heat shock protein (HSP), IL-2, adhesion molecules, mucin, sialomucin, metalloproteinases, other proteinases, monocyte colony stimulating factor (MCSF), platelet derived growth factor (PDGF), Angiotensin II, pregnancy associated plasma protein A (PAPPA) and chemoattractant peptide.

60. A method of treating Atherosclerosis, comprising administrating to a mammal in need of such treatment, at least one of the reagents of claim 59.

61. A pharmaceutical preparation for reducing of at least one molecular inflammatory factor (MIF) or cellular inflammatory factor (CIF), the pharmaceutical preparation comprising a species selected from a non-catalytic polyclonal antibody, a catalytic polyclonal antibody, a non-catalytic monoclonal antibody, a catalytic monoclonal antibody, antibody fragment, a synthetic antibody fragment, an antibody analog, a chimeric monoclonal antibody, a humanized monoclonal antibody, a fragment of any of the above antibodies, including synthetic fragments and analogs of such fragments, wherein said antibody, antibody fragment or analog selectively binds said MIF or CIF and including a pharmaceutically acceptable carrier.

62. The pharmaceutical preparation of claim 61, wherein at least one MIF comprises IL6.

63. A method of treating a mammal in need of treatment aimed at the reduction of at least one molecular inflammatory factor (MIF) or cellular inflammatory factor (CIF), the method comprising administering to the mammal the pharmaceutical preparation of claim 61.

64. A method of treating a mammal in need of treatment aimed at the reduction of interleukin 6 (IL6), by administering to the mammal the pharmaceutical preparation of claim 62.

65. The method of claim 63, further including the administration of an anti inflammatory cytokine.

66. The method of claim 65 wherein the anti inflammatory cytokine comprises at least one of the group comprising interleukin 10 (IL10) and TGF-Beta.

67. The method of claim 64, further including the administration of an anti inflammatory cytokine.

68. The method of claim 67 wherein the anti inflammatory cytokine comprises at least one of the group comprising interleukin 10 (IL10) and TGF-Beta.

69. The pharmaceutical preparation of claim 61 wherein the antibody is selected from the group consisting of a non-catalytic polyclonal antibody, a catalytic polyclonal antibody, a non-catalytic monoclonal antibody, a catalytic monoclonal antibody, an antibody fragment, a synthetic antibody fragment, and an antibody analog.

70. The pharmaceutical preparation of claim 69 wherein the monoclonal antibody comprises a non-humanized chimeric monoclonal antibody, a humanized monoclonal antibody, a fragment of any of the above antibodies, including synthetic fragments and analogs of such fragments.

71. The pharmaceutical preparation of claim 41 comprising an adjuvant.

72. The pharmaceutical preparation of claim 71 wherein the adjuvant is suitable for administering to humans.

73. The pharmaceutical preparation of claim 41 comprising a liposome.

74. The pharmaceutical preparation of claim 41 wherein the immunogenic preparation comprises a carrier.

75. The pharmaceutical preparation of claim 74 wherein the carrier is selected from KLH, Albumin and peptides.

76. An extracorporeal device, comprising at least one affinity adsorbent, said affinity adsorbent binding at least one endotoxin wherein the at least one affinity adsorbent is selected from antibodies, antibody fragments, synthetic antibody binding site analogs, genetically engineered synthetic antibody binding site analogs, endotoxin receptor binding sites, synthetic or genetically engineered endotoxin receptor binding site analogs.

77. The device of claim 76 wherein the affinity adsorbent is chemically bound to a matrix in said device.

78. The device of claim 76 wherein the device comprises at least two affinity adsorbents, said affinity adsorbents binding selectively to two different endotoxins.

79. The device of claim 76 further comprising means for regenerating at least one affinity adsorbent.

80. The device of claim 79 wherein the regeneration means comprises a solution.

81. The device of claim 80, wherein the solution comprises a buffer.

82. The device of claim 81 wherein the buffer is an acidic buffer.
